(19) United States
(12) Patent Application Publication
(10) Pub. No.: US 2022/0172847 A1
(43) Pub. Date: Jun. 2, 2022

(54) APPARATUS, SYSTEMS AND METHODS FOR PREDICTING, SCREENING AND MONITORING OF MORTALITY AND OTHER CONDITIONS UIRF 19054

(71) Applicant: Gen Shinozaki, Iowa City, IA (US)

(72) Inventor: Gen Shinozaki, Iowa City, IA (US)

(21) Appl. No.: 17/601,344

(22) PCT Filed: Apr. 6, 2020
(86) PCT No.: PCT/US20/26914

§ 371 Date: Oct. 4, 2021

Related U.S. Application Data
(60) Provisional application No. 62/829,411, filed on Apr. 4, 2019.

(51) Int. Cl. G16H 50/30; G16H 50/50; A61B 5/00; A61B 5/374; A61B 5/384; A61B 5/16

(52) U.S. Cl. CPC G16H 50/30; G16H 50/50; A61B 5/165; A61B 5/374; A61B 5/384; A61B 5/7275

(57) ABSTRACT

The disclosed apparatus, systems and methods relate to predicting, screening, and monitoring for mortality and other negative patient outcomes. Systems and methods may include receiving one or more signals from one or more sensing devices; processing the one or more signals to extract one or more features from the one or more signals; analyzing the one or more features to determine one or more values for each of the one or more features; comparing at least one of the one or more values or a measure based on at least one of the one or more values to a threshold; determining a presence, absence, or likelihood of the subsequent mortality, falls or extended hospital stays for a patient based on the comparison; and outputting an indication of the presence, absence, or likelihood of the subsequent development of poor outcomes or death for the patient.

### CROSS-REFERENCE

This application claims priority to International PCT Application No. PCT/US20/26914 filed on Apr. 6, 2020, which claims priority to U.S. Patent Application No. 62/829,411, filed Apr. 4, 2019, and entitled “Apparatus, Systems And Methods For Predicting, Screening And Monitoring Of Mortality And Other Conditions,” which is hereby incorporated herein by reference in its entirety.

### GOVERNMENT SUPPORT

This invention was made with government support under 1664364 Awarded by the National Science Foundation. The government has certain rights in the invention.

### TECHNICAL FIELD

Discussed herein are various devices, systems, and methods for use in medicine and particularly to medical devices.

### BACKGROUND

Delirium is an acute state of confusion characterized by inattention, impaired cognition, psychomotor disturbances, and a waxing and waning course. Delirium is particularly common in older, hospitalized adults affecting a significant number of patients on general medicine floors, postoperative procedure units including electroconvulsive therapy, and intensive care units.

Delirium in hospitalized elderly patients is common, dangerous, and expensive. It is also seriously underdiagnosed and therefore undertreated. It is estimated there are minimally 2-3 million cases of delirium per year in the US alone. Delirium is a strong predictor of poor patient outcomes. Delirium increases mortality, complications, hospital length of stay, and institutionalization after discharge. Even when these patients survive, they have a high risk of long-term cognitive impairment. If undetected, delirium can add thousands of dollars in healthcare costs per patient per year, creating billions of dollars in added healthcare costs.

Delirium is common and dangerous, yet under-detected and under-treated. Current screening questionnaires are subjective and ineffectively implemented in busy hospital workflows. Electroencephalography (EEG) can objectively detect the diffuse slowing characteristic of delirium, but it is not suitable for high-throughput screening due to size, cost, and the expertise required for lead placement and interpretation.

Relationship between delirium and dementia is often complicated because dementia is one of the risk factors of delirium. In addition, delirium is known to accelerate the progression of dementia. Furthermore, delirium and dementia are associated with patients' outcomes including mortality. Especially if patients have both delirium and dementia, their mortality would increase.

There is a need in the art for efficient and reliable devices, systems, and methods for predicting and screening for mortality.

### BRIEF SUMMARY

Discussed herein are various devices, systems and methods relating to systems, devices and methods for detecting, identifying or otherwise predicting mortality and/or other conditions in a patient. In various implementations, a device is utilized to detect diffuse slowing—a hallmark of these conditions.

The disclosed embodiments relates to systems and methods for predicting, screening, and monitoring of mortality or other conditions, and, more specifically, to systems and methods for determining the presence, absence, or likelihood of subsequent development of mortality or other conditions in a patient by signal analysis. Output data includes presenting an indication of risk for poor outcomes including mortality, extended hospital stay, institutionalization after discharge and the chance of a fall in the hospital. In various implementations, the output is continuous score, indicating the higher it is, the more likely patients have poor outcomes. In additional implementations, the disclosed systems, methods and devices include the execution of an intervention or treatment to prevent undesirable outcomes.

Systems and methods are described for using various tools and procedures for predicting, screening, and monitoring of mortality and other negative outcomes such as extended hospital stay, institutionalization after discharge and the chance of a fall in the hospital. In certain embodiments, the tools and procedures described herein may be used in conjunction with one or more additional tools and/or procedures for predicting, screening, and monitoring of mortality. The examples described herein relate to predicting, screening, and monitoring of mortality for illustrative purposes only. For multi-step processes or methods, steps may be performed by one or more different parties, servers, processors, and the like.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In Example 1, a method for patient screening for outcome risk, comprises recording raw BSEEG values via a handheld device, normalizing the raw BSEEG values to calculate a NBSEEG, and outputting an outcome NBSEEG score.

Example 2 relates to the method of Example 1, wherein the NBSEEG is calculated by comparing the raw BSEEG with a BSEEG population mean; and dividing the result by population by the BSEEG population standard deviation.

Example 3 relates to the method of Example 1, wherein the outcome NBSEEG score comprises an NBSEEG positive or NBSEEG negative score.

Example 4 relates to the method of Example 1, wherein the outcome NBSEEG score is continuous.

Example 5 relates to the method of Example 1, wherein the recording is performed at a primary point of care.

Example 6 relates to the method of Example 1, wherein the outcome NBSEEG is correlated with at least one of hospital length of stay (“LOS”), discharge disposition, and/or mortality risk.

In Example 7, a handheld system for patient screening for mortality risk, comprises at least two sensors configured to record one or more brain frequencies; a processor; and at least one module. The at least one module configured to record raw BSEEG values; normalize the raw BSEEG values to calculate a NBSEEG; output an outcome NBSEEG score.

Example 8 relates to the system of Example 7, wherein the outcome NBSEEG is correlated with at least one of hospital LOS, discharge disposition, and/or mortality risk.

Example 9 relates to the system of Example 7, further comprising outputting threshold data.

Example 10 relates to the system of Example 7, further comprising comparing the outcome NBSEEG score to a threshold.

Example 11 relates to the system of Example 7, further comprising a signal processing device.

In Example 12, a method of screening for mortality risk in a subject, comprises recording raw BSEEG values from the subject via a handheld device; normalizing the raw BSEEG values to calculate a NBSEEG; and outputting an outcome NBSEEG score.

Example 13 relates to the method of Example 12, further comprising comparing the outcome NSBEEG score to a threshold.

Example 14 relates to the method of Example 12, wherein the raw BSEEG values are processed via a signal processing module or feature analysis module in the handheld device.

Example 15 relates to the method of Example 12, wherein the outcome NBSEEG score is categorized as low, medium or high risk by comparison to one or more thresholds.

Example 16 relates to the method of Example 12, further comprising maintaining a BSEEG population norm.

Example 17 relates to the method of Example 16, wherein the NBSEEG is calculated by comparing the raw BSEEG with the mean of the BSEEG population norm; and dividing the result by population by the BSEEG population standard deviation.

Example 18 relates to the method of Example 17, further comprising recording subject outcome.

Example 19 relates to the method of Example 18, wherein the BSEEG population norm is updated to include the raw BSEEG values and subject outcome.

Example 20 relates to the method of Example 19, wherein the outcome NBSEEG is correlated with at least one of hospital length of stay (“LOS”) and/or discharge disposition.

In certain implementations, the disclosed Examples relate to a method for predicting mortality by recording an EEG score comprising a ratio of high and low frequency components. The EEG signal is recorded via a point-of-care, portable EEG device with a limited number of electrodes in certain implementations. In certain implementations, the raw EEG signals are processed by spectral density analysis, followed by an algorithm to combine low frequency power and high frequency power such as ratio between the two or more, to produce a raw BSEEG value.

Various Examples relate to assigning a normalized BSEEG outcome NBSEEG score (“NBSEEG score”) by dividing the difference between the raw BSEEG score and average of a BSEEG score population norm by the standard deviation of a BSEEG population norm. In certain implementations, the raw BSEEG value is assessed compared to a population BSEEG score distribution in a relationship to its average and standard deviation. The population mean can be defined by certain patient groups or healthy population group. The NBSEEG score according to certain implementations is obtained by (Raw BSEEG value−population norm BSEEG average) divided by the standard deviation of BSEEG from the population norm.

Various Examples involve outputting the resulting outcome NBSEEG score into one of two, three or more different levels of outcomes such as morality outcomes. The NBSEEG score can be used as a continuous value as a new vital sign, just like body temperature, blood pressure and heart rate. The risk threshold for mortality is thresholded via an ongoing risk score that is defined via epidemiological study. The data presented in the present disclosure showed that high NBSEEG score can lead higher mortality, and low NBSEEG score can have less risk. When the score was divided into three groups, the score showed dose dependent relationship to the mortality risk.

One general aspect includes a system for patient screening, including a handheld screening device including a housing; at least two sensors configured to record one or more brain signals and generate one or more values; a processor and at least one module configured to: perform spectral density analysis on the one or more values and output data presenting an indication of the presence, absence, or likelihood of the subsequent development of mortality. Other embodiments of this aspect include corresponding computer systems, apparatus, and computer programs recorded on one or more computer storage devices, each configured to perform the actions of the methods.

Implementations may include one or more of the following features. The system where the module is configured to compare one or more values from the one or more brain signals to a threshold. The system where the threshold is a ratio including a number of occurrences of high frequency waves to a number of occurrences of low frequency waves. The system where the one or more brain signals are electroencephalogram (EEG) signals. The system where there are two sensors. The system where the housing includes a display. The system where the processor is disposed within the housing. The system where the one or more values are selected from the group including of: high frequency waves, low frequency waves, and combinations thereof. The system where the one or more values are numeric representations of the number of occurrences of each of the one or more features over a period of time. The system where the threshold is predetermined. The system where the threshold is established on the basis of a machine learning model. The system further including a handheld housing including a display, where: at least two sensors are in electronic communication with the housing, the processor is disposed within the housing, and the display is configured to depict the output data. The system further including a validation module configured to evaluate brain signal, where the processor converts the one or more brain frequencies into signal data, and the validation module discards the signal data that exceeds at least one pre-determined signal quality threshold. The system where the signal data is partitioned into windows of equal duration. The device further including a signal processing module. The device further including a validation module. The device further including a threshold module. Implementations of the described techniques may include hardware, a method or process, or computer software on a computer-accessible medium.

One general aspect includes a system for evaluating the presence of mortality risk, including: a. at least two sensors configured to record one or more brain frequencies; a processor; at least one module configured to: compare brain wave frequencies over time; perform spectral density analysis on the brain wave frequencies to establish a ratio; compare the ratio against an established threshold; and output data presenting an indication of the presence, absence, or likelihood of the subsequent development of mortality. Other embodiments of this aspect include corresponding computer systems, apparatus, and computer programs recorded on one or more computer storage devices, each configured to perform the actions of the methods.

Implementations may include one or more of the following features. The system where the threshold is predetermined. The system where the threshold is established on the basis of a machine learning model. The system further including a handheld housing including a display, where: the at least two sensors are in electronic communication with the housing, the processor is disposed within the housing, and the display is configured to depict the output data. The system further including a validation module configured to evaluate signal brain, where the processor converts the one or more brain frequencies into signal data, and the validation module discards the signal data that exceeds at least one pre-determined signal quality threshold. The system where the signal data is partitioned into windows of equal duration. The device further including a signal processing module. The device further including a validation module. The device further including a threshold module. Implementations of the described techniques may include hardware, a method or process, or computer software on a computer-accessible medium.

One general aspect includes a handheld device evaluating the presence, absence, or likelihood of the subsequent development of mortality in a patient, including: a housing; at least one sensor configured to generate at least one brain wave signal; at least one processor; at least one system memory; at least one program module configured to perform spectral density analysis on at least one brain wave signal and generate patient output data; and a display configured to depict the patient output data. Other embodiments of this aspect include corresponding computer systems, apparatus, and computer programs recorded on one or more computer storage devices, each configured to perform the actions of the methods.

Implementations may include one or more of the following features. The device further including a signal processing module. The device further including a validation module. The device further including a threshold module. The device further comprising a feature analysis module. The device further comprising a signal processing module. Implementations of the described techniques may include hardware, a method or process, or computer software on a computer-accessible medium.

One or more computing devices may be adapted to provide desired functionality by accessing software instructions rendered in a computer-readable form. When software is used, any suitable programming, scripting, or other type of language or combinations of languages may be used to implement the teachings contained herein. However, software need not be used exclusively, or at all. For example, some embodiments of the methods and systems set forth herein may also be implemented by hard-wired logic or other circuitry, including but not limited to application-specific circuits. Combinations of computer-executed software and hard-wired logic or other circuitry may be suitable as well.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1A shows an overview of an exemplary screening system in use on a patient.

FIG. 2A shows an example of diffuse slowing in brainwaves as would appear on an electroencephalogram.

FIG. 2B shows example normal conditions in brainwaves as would appear on an electroencephalogram.

FIG. 3A shows an example of diffuse slowing in brainwaves as would appear on an electroencephalogram.

FIG. 3B shows an example of two channel diffuse slowing in brainwaves as would appear on an electroencephalogram.

FIG. 3C shows an example of two channel normal condition brainwaves as would appear on an electroencephalogram.

FIG. 4A shows an exemplary system for predicting, screening, and monitoring of mortality risk.

FIG. 4B shows an exemplary system for computation aspects of predicting, screening, and monitoring of mortality risk

FIG. 5A shows an exemplary system for the predicting, screening, and monitoring of brain signals high risk for poor outcomes including mortality.

FIG. 5B shows an exemplary system for the predicting, screening, and monitoring of brain signals high risk for poor outcomes including mortality.

FIG. 5C shows an exemplary system for the predicting, screening, and monitoring of brain signals high risk for poor outcomes including mortality.

FIG. 5D shows another exemplary system for the predicting, screening, and monitoring of brain signals high risk for poor outcomes including mortality.

FIG. 6 shows program modules and program data of a screening device, according to an exemplary embodiment.

FIG. 7 is an overview of a method for predicting, screening, and monitoring of brain signals high risk for poor outcomes including mortality, according to an exemplary embodiment.

FIG. 8 is an overview of a method for predicting, screening, and monitoring of brain signals high risk for poor outcomes including mortality, according to an exemplary embodiment.

FIG. 9A shows an example of raw electroencephalogram signals.

FIG. 9B shows an example of raw electroencephalogram signals.

FIG. 9C shows an example of spectral density analysis.

FIG. 9D shows an example of spectral density analysis.

FIG. 10A shows an overview of an exemplary screening system in use on a patient.

FIG. 10B is a model flow chart for predicting outcome BSEEG scores, according to one embodiment.

FIG. 11. Distribution of Normalized BSEEG (NBSEEG) Scores. Scores are based on a total of 2938 recordings from 428 patients of all age groups. The NBSEEG score is defined as the number of standard deviations from the mean, with the mean being a score of 0.

FIG. 12. Study participant Enrollment Flow Chart. The study population was categorized in two ways: clinical delirium status and BSEEG score.

FIG. 13. Survival curve over 360 days based on clinical delirium status (left panel) and NBSEEG score (right panel). Patients who were clinically delirious had increased mortality compared to those without clinical delirium (P=0.0038). NBSEEG positive patients showed higher mortality than NBSEEG negative patients, regardless of clinical delirium status (P=0.0032).

FIG. 14. Survival curve over 360 days based on three NBSEEG categories. Mortality was directly proportional to the NBSEEG score, with higher NBSEEG score group associated with higher mortality (P=0.005).

FIG. 15. Subgroup analysis of mortality based on both clinical delirium status and NBSEEG category. Patients who were both clinical delirious and NBSEEG positive showed the highest mortality (purple line). Patients who were clinically delirious but NBSEEG negative had lower mortality (blue line), almost as low as patients who were both clinically non-delirious and NBSEEG negative (orange line). In contrast, patients who were clinically non-delirious, but NBSEEG positive, had higher mortality rates (green line), indicating that NBSEEG score was a better predictor of mortality than clinical delirium status.

FIG. 16. Predictive model of diagnosis based on EEG features using Random Forests (RF). Predictive power of individual genera (EEG features) of clinical status assessed by Boruta feature selection algorithm. The three horizontal lines of the box represent the first, second (median) and third quartiles respectively with the whisker extending to 1.5 inter-quartile range (IQR). Blue boxplots correspond to minimal, average and maximum Z score of shadow genera, which are shuffled version of real genera introduced to RF classifier and act as benchmarks to detect truly predictive genera. Red, yellow and green colors represent rejected, suggestive and confirmed genera by Boruta Selection. Here, importance is defined as the mean decrease in classification accuracy.

FIG. 17A-D. Survival Curve in 180 Days Based on the NBSEEG Category: A. 2 NBSEEG Categories in 228 subjects (replication cohort); B. 3 NBSEEG Categories in 228 subjects (replication cohort); C. 2 NBSEEG Categories in 502 subjects (discovery and replication cohorts); and D. 3 NBSEEG Categories in 502 subjects (discovery and replication cohorts). Abbreviations: NBSEEG, bispectral electroencephalography; B (−), NBSEEG Negative; B (+), NBSEEG Positive.

FIG. 18. Survival Curve in 180 Days Based on the Dementia and NBSEEG Categories. Abbreviations: NBSEEG, normalized bispectral electroencephalography; B (−), NBSEEG Negative; B (+), NBSEEG Positive; D (−), Dementia Negative; D (+), Dementia Positive.

FIG. 19A-C. Short-term Mortality Based on the NBSEEG Category in 502 subjects (discovery and replication cohorts): A. 30 Days; B. 60 Days; and C. 90 Days. Notes: * Relative risk was significantly higher than those in NBSEEG negative group. Abbreviation: NBSEEG, normalized bispectral electroencephalography.

FIG. 20. Short-term Mortality Based on the Dementia and NBSEEG Categories in 502 subjects (discovery and replication cohorts). * Relative risk was significantly higher than those in NBSEEG negative group. Abbreviation: NBSEEG, normalized bispectral electroencephalography.

### DETAILED DESCRIPTION

The various embodiments disclosed or contemplated herein relate to systems, methods and devices able to provide objective clinical measurements of mortality risk. These implementations detect the presence of diffuse slowing in the brain waves of patients. The implementations discussed herein are able to detect diffuse slowing by performing a spectral density analysis on brain waves recorded from a small number of discrete locations on the head of the patient, thereby enabling easier bedside diagnosis, such as with a handheld device. That is, the various implementations are able to record a brain waves via two or more leads placed on the head of a patient, and execute an algorithm to evaluate the ratio of recorded low frequency to high frequency waves and compare that ratio against a determined threshold to identify the risk of mortality. In further embodiments, these implementations utilize machine learning and additional data, such as that from medical records, to improve diagnostic accuracy.

The disclosed normalized bispectral electroencephalography (“NBSEEG”) method, systems and devices can also predict patient outcomes, including hospital length of stay, discharge disposition, and mortality from NBSEEG score obtained on the first day of their hospital stay. Brain signals are obtained from forehead from patients, and a novel algorithm used to calculate raw BSEEG value data, which is compared to mass data from ˜3,000 raw BSEEG value recordings from patients, to provide a normalized BSEEG (NBSEEG) score. When the score is high, it is associated with longer hospital stay, higher likelihood of discharge NOT to home, and higher mortality. The described implementations can be used to screen large volume of patients and provide objective score to predict patient outcomes, thus early intervention can be possible to improve patient outcomes.

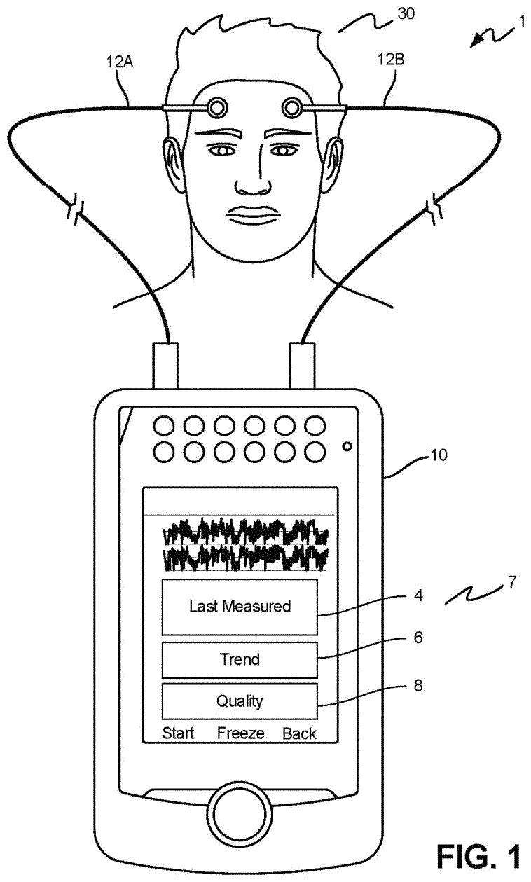

The disclosed systems, devices and methods relate to non-invasive, point of care diagnostics using fewer than the sixteen-, twenty- or twenty four-lead EEGs found in the prior art. For example, as shown generally in FIG. 1, in certain implementations a 2-lead BSEEG screening system 1 is employed, which can be performed with a handheld screening device 10 by applying two leads 12A, 12B to the forehead of a patient 30 for less than 10 minutes. While in these implementations, two leads or channels in the BSEEG are used for purposes of explanation, it is understood that many numbers of leads or channels are contemplated herein. In various implementations, the device 10 is able display graphical and/or numerical representations 7 of useful information for use in prediction of mortality, brain dysfunction, and/or extended hospital stays and risks associated therewith, such as the last measured value 4, the trend 6, signal quality 8 and the like. It is understood that these representations 7 can be the result of understood graphical user interface techniques on the display 16.

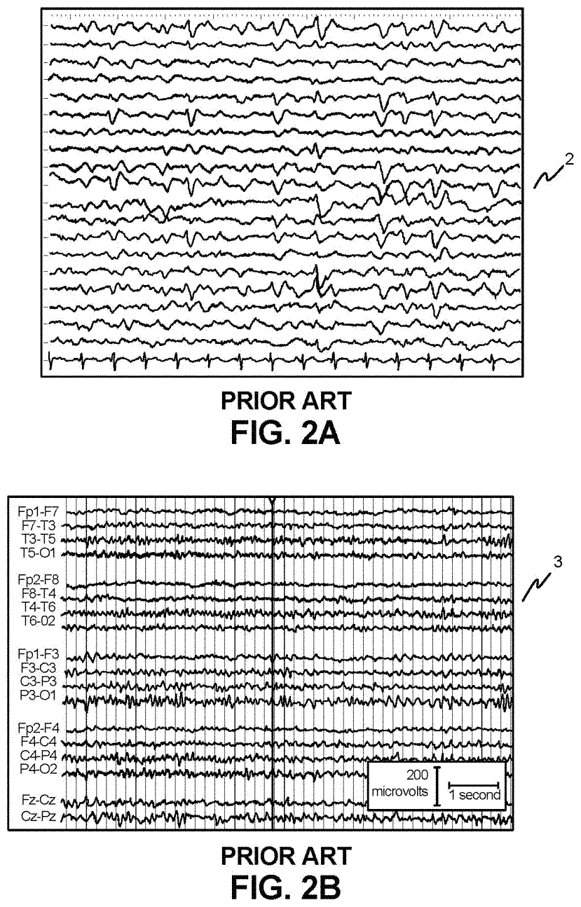

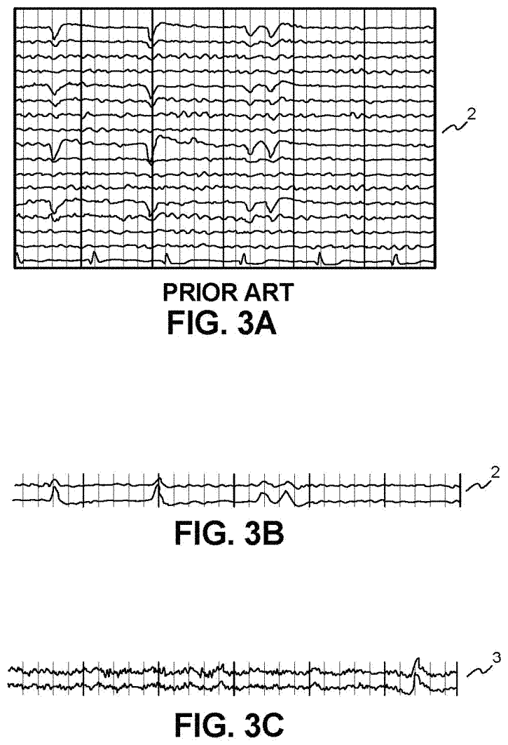

Brain waves may have various frequencies and/or bands of frequencies. “Diffuse slowing” is a strong predictor of mortality. FIGS. 2A-3D depict several EEG readings from patients experiencing symptoms of mortality 2, as compared to normal controls 3. As would be apparent to one of skill in the art, in various states, brain waves in mortality may be characterized by “diffuse slowing,” 2 meaning that slowed waveforms can be observed on each of the channels observed. As is apparent from FIGS. 2A and 3A, because this slowing is diffuse, rather than localized, the slowing (shown in FIGS. 2A and 3A) is observed at most—and typically all—of the various electrodes of an EEG.

As shown in FIG. 3A, emergence of slower waves 2 as compared to the number of higher frequency waves may be an indication that a patient has or is more likely to die or experience the other negative medical outcomes described herein.

As shown in FIG. 3B, because diffuse slowing can be routinely observed at all or nearly all of the EEG electrodes, it is possible to utilize fewer than the standard number of sixteen to twenty four EEG channels to identify diffuse slowing and therefore predict the risk of mortality. In these implementations, the two leads utilized in the BSEEG implementations discussed herein may be adequate for detecting diffuse slowing and, with appropriate signal processing and user interface, may require no special expertise for placement or interpretation, and may be performed with the aid of a simple handheld screening device. To quantify the risk of mortality, raw BSEEG value is compared to an established population norm distribution to accurately calculate normalized BSEEG (NBSEEG) score, which may be expressed as NBSEEG positive (BSEEG(+)) or NBSEEG negative (BSEEG(−)).

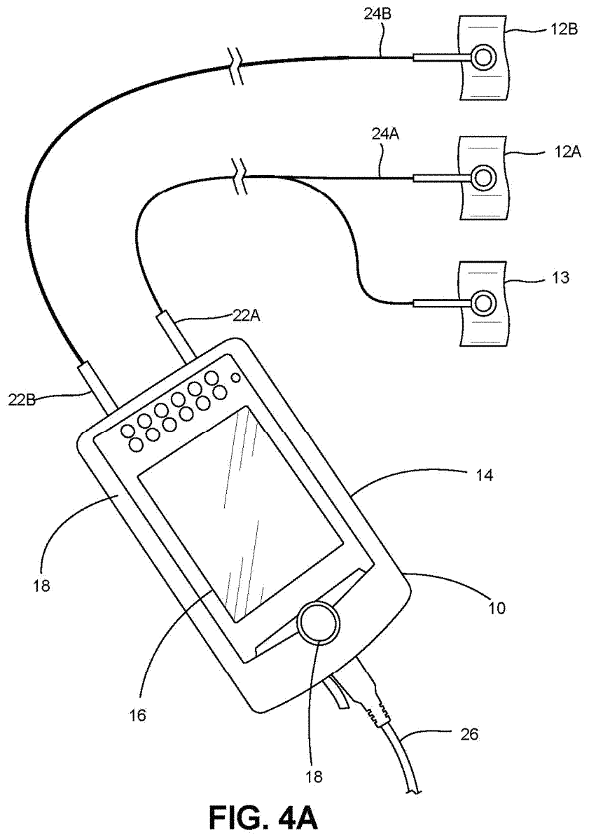

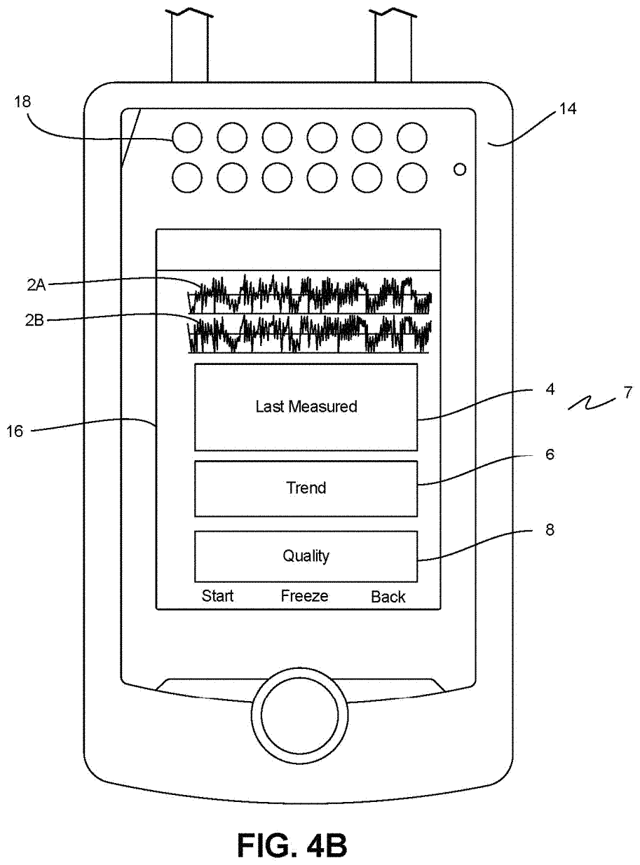

One implementation of a screening device 10 is shown in FIG. 4A. In various implementations, the systems and methods for predicting, screening, and monitoring of mortality disclosed herein may utilize such a handheld or otherwise portable screening device 10. In these implementations, the screening device 10 is configured to receive signals from, for example, one or more sensors 12A, 12B. Because diffuse slowing is readily identifiable across the brain of the patient, these devices 10 are able to use far fewer than twenty sensors, such as two, three, four, five or more sensors 12. In certain implementations, between six and twenty or more sensors are used. It is therefore understood that because fewer than twenty sensors 12 are used, the disclosed devices and systems are able to be easily transported and used on a patient 30, as there is no need to apply a typical prior art EEG cap.

In the implementation of FIG. 4A, the one or more sensors 12A, 12B are brain sensors, such as, but not limited to, electrodes placed on a patient. In certain embodiments, the signals may be electroencephalograph (“EEG”) signals from the one or more electrodes measuring brain activity of a patient. The signals may be processed to extract one or more features of the signals. The one or more features may be analyzed to determine one or more values for each of the one or more features. The values, or a measure based on one or more of the values, may be compared against a threshold to determine the presence, absence, or likelihood of subsequent development of mortality if the patient does not currently exhibit clinical signs or symptoms of the disease.

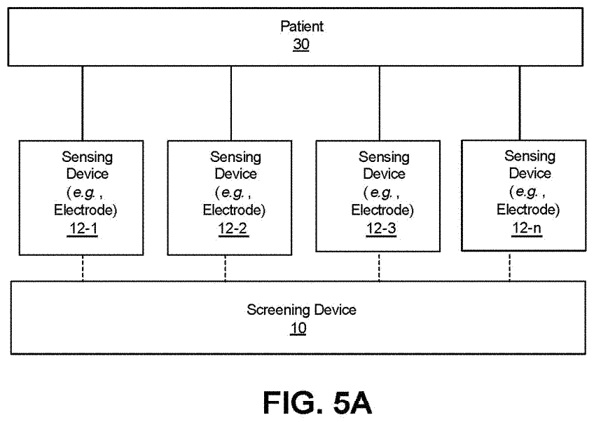

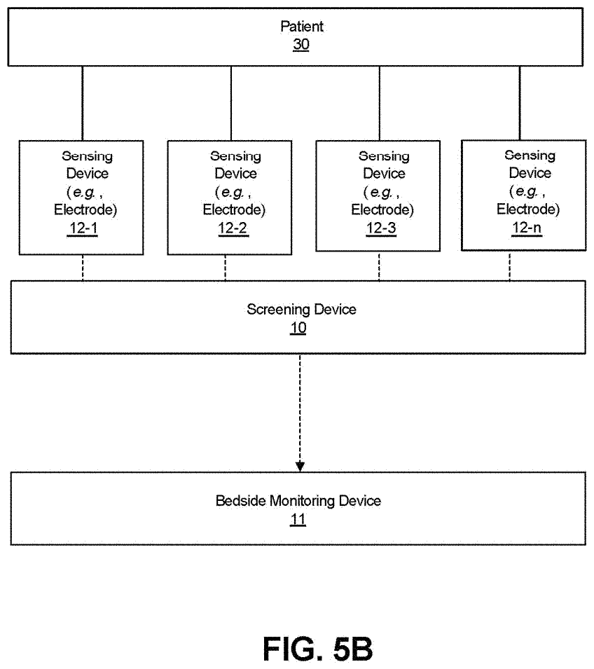

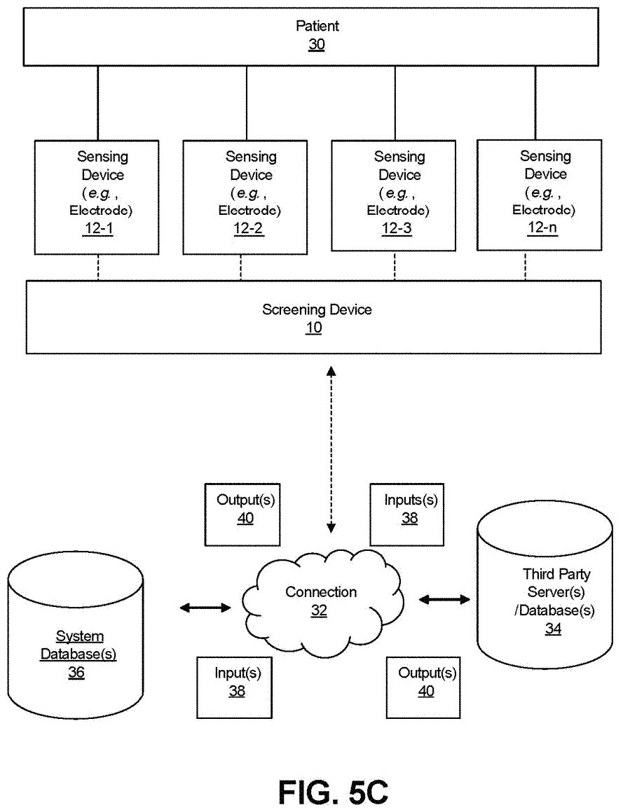

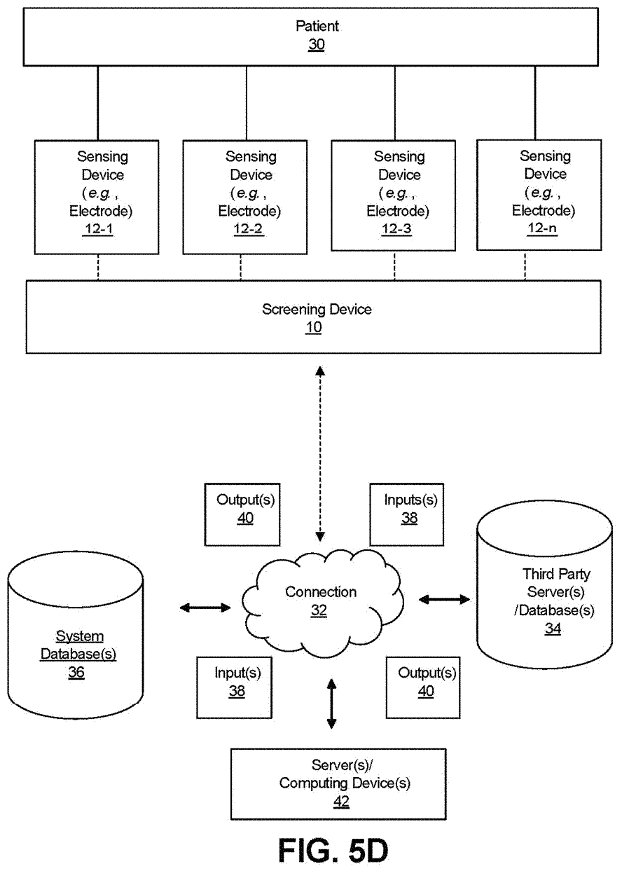

Continuing with FIGS. 4A-4B, the screening device 10 can comprise a housing 14. In various implementations, the housing 14 can be sized to be handheld or otherwise portable. The housing 14 can have a display 16 and interface 18, such as buttons or a touch screen, as would be appreciated by the skilled artisan. In various implementations, the sensors 12A, 12B are connected to the device 10 via ports 22A, 22B and wires 24A, 24B, while alternate implementations utilize a wireless interface such as a Bluetooth® or the like. As shown in FIG. 4A, a ground lead 13 can also be provided, which can be bundled with one of the wires 24A, 24B to streamline application. In various implementations, a transmission cord 26 or other connection can be used to place the device 10 in electrical communication with a server or other computing device, as is discussed in relation to FIGS. 5A-B.

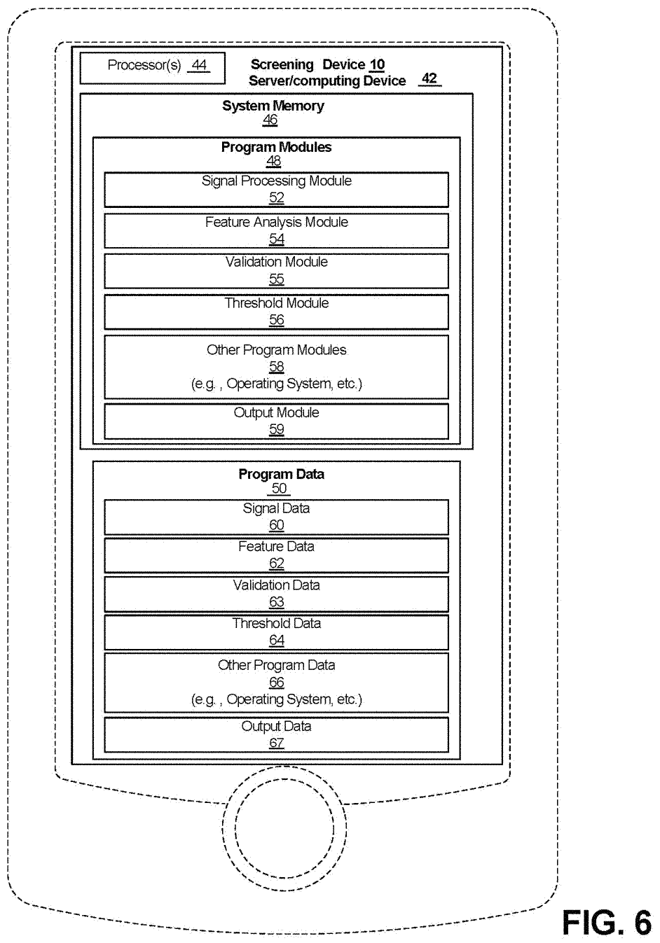

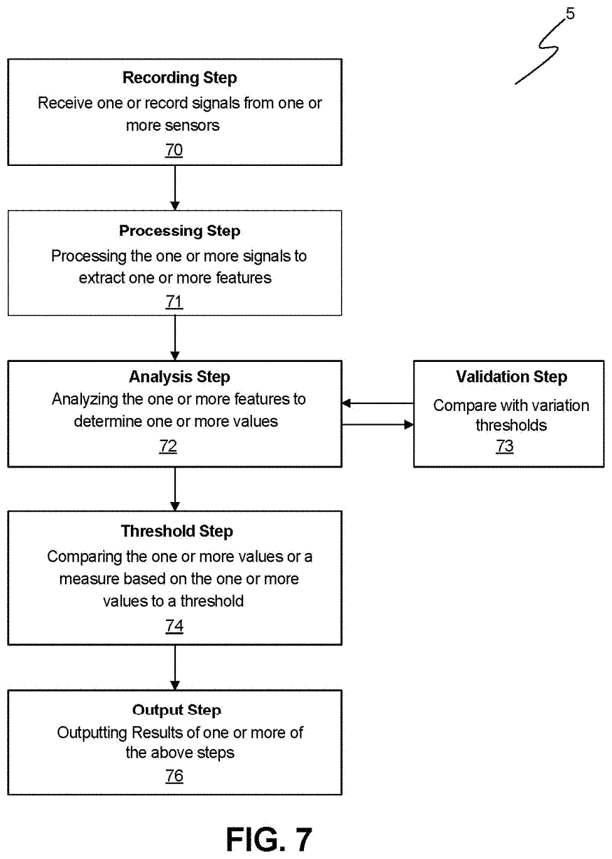

As shown in FIG. 4B, in various implementations the display 16 can depict graphical and/or numerical representations 7 of useful information for use in diagnosis of mortality and/or delirium including at least one of: one or more brain waves 2A, 2B, the last measured value 4, the trend 6, signal quality 8 and the like. In exemplary implementations, a graphical representation of the threshold step described below, which compares the spectral density with an established threshold as shown as the last measured value 4. It is understood that these representations 7 can include any program data 67, and can be shown to the provider as the result of understood graphical user interface techniques on the display 16. In these embodiments, the brain waves 2A, 2B are drawn from the sensors 12 placed on the patient, and can be used to diagnose and/or predict the onset of mortality in a clinical or other setting. In various implementations, the housing 14 also comprises a power supply and computing components, such as a microprocessor, memory and the like. In additional implementations, computing and other processing is done within the device housing 14, such as via program modules (described in relation to FIGS. 6-7), while additional processing can be performed elsewhere, as described herein. In each of these implementations, the screening device 10 and/or system 1 comprises a processor configured to evaluate diffuse slowing in the patient by way of spectral density analysis performed on fewer than 20 channels to identify diffuse slowing.

FIG. 5A shows an exemplary system 1 for the predicting, screening, and monitoring of mortality according to one implementation. In this implementation, the system 1 may include one or more screening devices 10 (e.g., screening device 1, screening device 2, . . . , screening device n), which in certain implementations is the device 10 of FIG. 1. The screening devices 10 in this implementation are operatively coupled to the one or more sensors 12-1 to 12-n. The one or more sensors 12 may be individual sensors, arrays of sensors, medical devices, or may be other computing devices, remote access devices, etc. In certain embodiments, the one or more sensors 12 may be one or more electrodes and/or other brain function monitoring devices. The one or more sensors 12 may be directly or indirectly coupled to a patient 30 for monitoring biological signals of the patient 30. In certain embodiments, certain of the one or more sensors 12 may be directly coupled to and/or integral with the screening device 10 via the ports 22 (best shown in FIG. 4A).

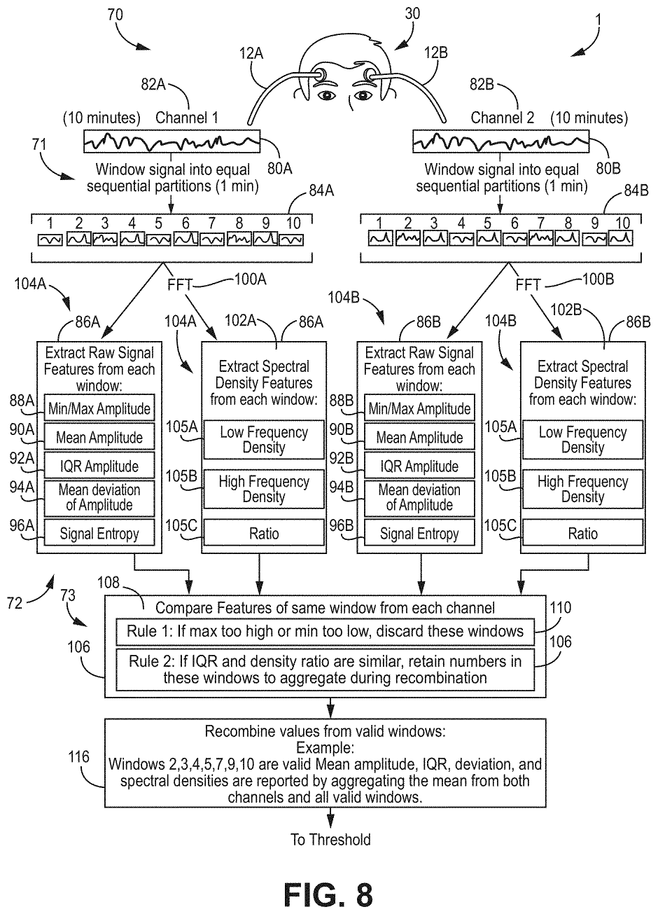

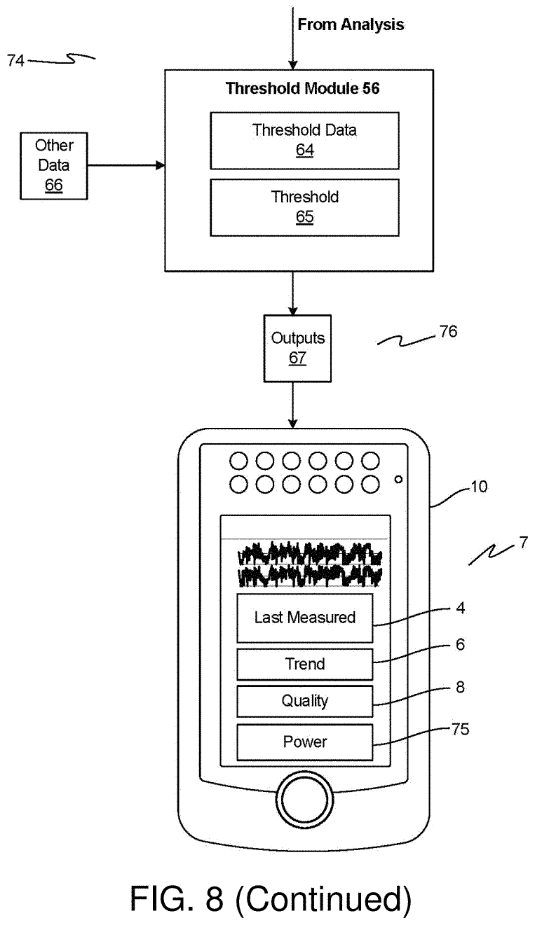

As also shown in FIG. 5A, various processing and analysis of the signals received from the sensors 12-1 12-2, 12-3, 12-n can be performed on the screening device 10, as are described in relation to FIGS. 6 and 8. In certain alternate implementations, such as those shown in FIGS. 5B-D, the screening device 10 can be used in conjunction with other computing devices.

As shown in the implementation of FIG. 5B, the screening device can be connected or otherwise interfaced to a bedside device 11. In various implementations, the bedside device 11 can be a patient monitor or other unified bedside monitoring device 11. In various implementations, the bedside device 11 can be used by a healthcare provider to view and/or perform certain analysis or observation steps.

As shown in FIGS. 5C-5D, the one or more screening devices 10 may also be operatively connected directly and/or indirectly, such as over a network, to one or more servers/computing devices 42, system databases 36 (e.g., database 1, database 2, . . . , database n). Various devices may be connected to the system, including, but not limited to, medical devices, medical monitoring systems, client computing devices, consumer computing devices, provider computing devices, remote access devices, etc. This system 1 may receive one or more inputs 38 and/or one or more outputs 40 from the various sensors, medical devices, computing devices, servers, databases, etc.

As is shown in FIG. 5D in certain embodiments, the one or more screening devices 10 may be directly coupled to and/or integral with the one or more servers/computing devices 42 via a connection 32 and/or may be coupled to the one or more servers/computing devices 42 over one or more network connections 32. The screening device 10 and/or one or more servers/computing devices 42 may also be operatively connected directly and/or indirectly, such as over a network, to one or more third party servers/databases 34 (e.g., database 1, database 2, . . . , database n). The one or more servers/computing devices 42 may represent, for example, any one or more of a server, a computing device such as a server, a personal computer (PC), a laptop, a smart phone, a tablet or the like.

In various implementations, the connection 32 may represent, for example, a hardwire connection, a wireless connection, any combination of the Internet, local area network(s) such as an intranet, wide area network(s), cellular networks, Wi-Fi networks, and/or so on. The one or more sensors 12, which may themselves include at least one processor and/or memory, may represent a set of arbitrary sensors, medical devices or other computing devices executing application(s) that respectively send data inputs to the one or more screening devices 10 or servers/computing devices 42 and/or receive data outputs from the one or more screening devices 10 or servers/computing devices 42. Such servers/computing devices 42 may include, for example, one or more of desktop computers, laptops, mobile computing devices (e.g., tablets, smart phones, wearable devices), server computers, and so on. In certain implementations, the input data may include, for example, analog and/or digital signals, such as from an EEG system, other brain wave measurements, etc., for processing with the one or more servers/computing devices 10.

In various implementations, the data outputs 40 may include, for example, medical indications, recommendations, notifications, alerts, data, images, and/or so on. Embodiments of the disclosed embodiments may also be used for collaborative projects with multiple users logging in and performing various operations on a data project from various locations. Certain embodiments may be computer-based, web-based, smart phone-based, tablet-based and/or human wearable device-based.

In another exemplary implementation, the screening device 10 (and/or server/computing device 42) may include at least one processor 44 coupled to a system memory 46, as shown in FIG. 6. The system memory 46 may include computer program modules 48 and program data 50. As described above, the operations associated with respective computer-program instructions in the program modules 48 could be distributed across multiple computing devices.

Spectral density analysis from the leads can be performed by a variety of electronic and computing mechanisms. In the implementation of FIG. 6, program modules 48 are provided. These program modules may include a signal processing module 52, a feature analysis module 54, a validation module 55, a diffuse slowing or mortality determination, threshold module 56, an output module 59, and/or other program modules 58 such as an operating system, device drivers, etc. In various implementations, each program module 52 through 58 may include a respective set of computer-program instructions executable by processor(s) 44.

FIG. 6 depicts one example of a set of program modules and other numbers and arrangements of program modules are contemplated as a function of the particular arbitrary design and/or architecture of server/computing device 10 and/or system 1. In an exemplary implementation, program data 50 may include signal data 60, feature data 62, validation data 63, diffuse slowing or mortality determination module 64, output data 67 and other program data 66 such as data input(s), third party data, and/or others configured to implement the various steps described in FIGS. 7-8.

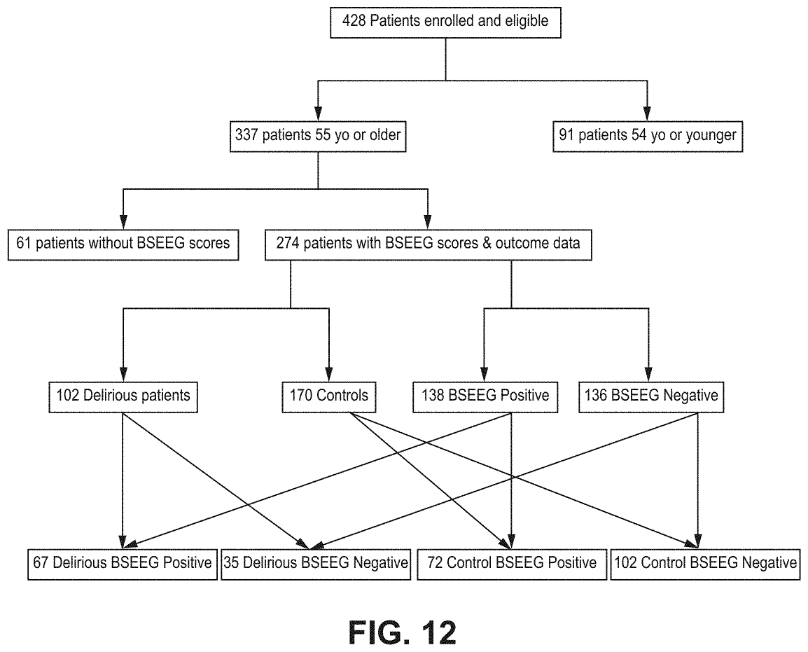

In various implementations, program data 50 may correspond to the various program modules 48 discussed above. In these various implementations, the program modules 48 and/or program data 50 can be used to record, analyze, and otherwise product output data 67 to the display (as shown at 16 in FIGS. 1A and 8) and/or any of the other computing devices described herein, such as the bedside monitoring device 11 of FIG. 5B, the various system databases 36 and/or third party servers or databases 34 of FIG. 5C, the system server(s)/computing device(s) 42 of FIG. 5D—or, in implementations where the module is not contained within the housing 14, to the screening device itself 10—as well as the gateway 210 of FIG. 12 or any other computing, storage or communications device in electrical or physical communication with the output module. It is understood that the output data 67 provides the point of care provider with characteristics of the patient brain waves for the purposes of diagnosing or otherwise identifying the symptoms of delirium or mortality. For example, in certain implementations, the provider can be provided with the last measured readings of the spectral density as well as qualitative measurements of the signal quality and trend, as has been describe elsewhere herein.

FIG. 7 is an overview of an exemplary method 5 according to an embodiment for predicting, screening, and monitoring of mortality and establishing a NBSEEG predictive outcome NBSEEG score. To achieve predicting, screening, and monitoring of mortality, the systems and methods may perform several optional steps.

One optional step is a recording step 70. In this step, input data, such as one or more raw BSEEG value input signals may be received and/or recorded by one or all of the sensors 12, device 10, processor(s) 40 and/or system memory 46 shown, for example, in FIGS. 4A-4B, 5A-5D and 6.

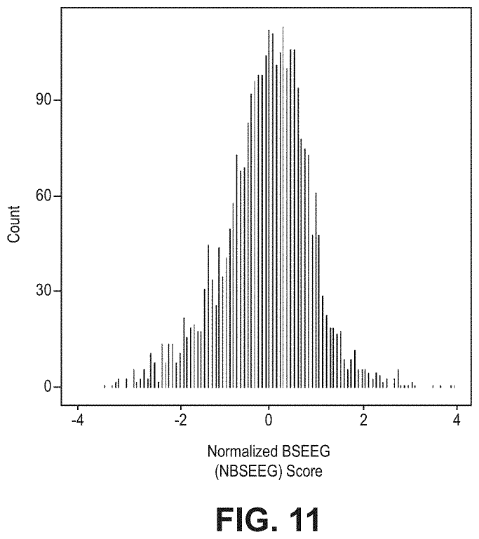

Continuing with FIG. 7, in an optional processing step 71, the one or more signals may be processed, such as by program data 50 and/or the signal processing module 52 shown in FIG. 6. Further, these signals can be processed to partition the signals into windows, as is also shown in FIG. 8. The signals can also be processed to extract one or more values from the one or more signals and/or windows, as is described further in relation to FIG. 8A. These values can include features of the raw BSEEG signal values from an EEG in certain implementations. In various implementations, the system 1 is able to trend these values to improve the accuracy of the system, as is described further in relation to FIGS. 11A and 11B.

Continuing with FIG. 7, an optional analysis step 72 can be performed, where one or more values may be analyzed to determine certain features of the signals or windows. In certain implementations, this analysis step can comprise performing a Fast Fourier Transform 100 to create or otherwise compare feature data (as shown in FIG. 6 at 64).

In certain implementations, the signal processing module 52 and/or feature analysis module 54 is configured to normalize raw BSEEG values in an analysis step 72, such as by taking the difference of the recorded BSEEG values from a population mean or threshold and then dividing by the standard deviation of the BSEEG population norm or threshold to establish a normalized BSEEG (NBSEEG) score. When a threshold is used, NBSEEG can be categorized as NBSEEG positive (NBSEEG(+)) or NBSEEG negative (NBSEEG(−)) scores, as is described in the below Examples.

In another optional step, a validation step 73 can be performed. In these implementations, and as shown in FIG. 8, the spectral density 102 and/or other raw BSEEG signal values 104A, 104B or other features can be used to compare the individual readings from the partitioned signal windows (shown generally at 84A, 84B) for inclusion or rejection from use in subsequent steps. In various implementations, this step can be performed by way of a validation module 55 and validation data 63. In various implementations, the validation step 73 can be performed concurrently or otherwise in repetitive iterations with the other steps described herein and utilize an error collection algorithm until the resultant signal data is ready for subsequent analysis. For example, and as described further in FIG. 8, the validation step 73 can be used to ensure that all readings above or below certain determined predetermined error values have been excluded from further processing.

Continuing with FIG. 7, a diffuse slowing or mortality determination step, or threshold step 74 can be performed. In this step, at least one of the one or more values or features or a measure based on at least one of the one or more values may be compared to an established diffuse slowing threshold (shown in FIG. 8 at 65), which can be implemented via diffuse slowing threshold data (as shown in FIG. 6 at 64). In certain embodiments, a presence, absence, or likelihood of the subsequent development of a negative outcome such as extended hospital stay, discharge to not home or mortality may be determined for a patient based on the threshold comparison. For example, and as discussed in the Examples below, using the mean of 3 Hz/10 Hz readings as the threshold data 64 and a threshold 65 of 1.44 (positive >=1.44 and negative <1.44), the screening device 10 may graphically indicate positive and/or negative readings, with or without the threshold 65 also being displayed. It is understood that the threshold 65 may also be modified over time as a result of system improvements and enhancements, as well as on the basis of additional data 66 and other factors.

In exemplary implementations, a graphical representation of the threshold step is displayed on the screening device 10 or other monitoring system, showing the comparison of the spectral density with the established threshold is shown as the last measured value 4. Each of these optional steps is discussed in further detail below in relation to the presently disclosed examples.

As shown in FIG. 8, in one exemplary implementation of the system 1, a patient 30 is being monitored by sensors 12A, 12B such as by way of the screening device 10 of FIG. 1. In this implementation, the sensors 12 produce one or more signals 80A, 80B from the patient 30. The one or more signals 80A, 80B may be analog and/or digital signals. The one or more sensors 12A, 12B may be separate from the system receiving the one or more signals, or may be integral with the system receiving the one or more signals. It is understood that while the presently described system 1 relates to the collection of EEG signals, in alternate implementations the one or more sensors 12A, 12B may measure, detect, determine, and/or monitor one or more of the following physiological conditions: heart rate, pulse rate, EKG, heart variability, respiratory rate, skin temperature, motion parameters, blood pressure, oxygen level, core body temperature, heat flow off the body, galvanic skin response (GSR), electromyography (EMG), electrooculography (EOG), body fat, hydration level, activity level, oxygen consumption, glucose or blood sugar level, body position, pressure on muscles or bones, and ultraviolet (UV) radiation absorption, etc.

In various implementations, and as discussed in relation to FIGS. 4A-6, the system 1 includes one or more signal processing devices, which can be disposed within the screening device 10 or elsewhere. The processing may determine the one or more features by parsing the one or more signals to look for signal information that corresponds to certain signal features. In certain embodiments, the one or more signals may be processed to determine the presence of one or more high frequency waves and/or one or more low frequency waves. The one or more features may be types of waves, such as, for example, but not limited to, high frequency waves and/or low frequency waves.

In the implementation of FIG. 8, the signals 80A, 80B are received through first 82A and second 82B channels for processing, as described above. Other implementations are possible. The methods may be integrated into 8-bit or 16-bit embedded device environments, or more robust environments as well. The methods may be integrated into existing hospital patient workflows.

As also shown in FIG. 8, in certain implementations the signals 80A, 80B can be processed to window the signal into equal sequential partitions (shown generally at 84A and 84B). In the depicted implementation, a signal duration of ten minutes is shown, wherein the signal is windowed into ten individual one minute windows (also shown generally at 84A and 84B), however it is understood that in alternative implementations other signal durations and numbers of windows can be used. In various implementations, the signals may be less than a second long or longer than an hour, or any number of minutes. Similarly, there can be any number of windows, and the windows can range in time from fractions of a second to many minutes or more.

In the implementation of FIG. 8, after the signals 80A, 80B have been partitioned, several optional processing and analysis steps can be performed, as described in relation to FIG. 7. In various implementations, the values 86A, 86B are extracted, and various optional steps can be performed to values 86A, 86B to identify raw data features 104A, 104B such that some or all of the minimum/maximum amplitude 88A, 88B, the mean amplitude 90A, 90B, the interquartile range (IQR) amplitude 92A, 92B, the mean deviation of amplitude 94A, 94B and/or signal entropy 96A, 96B can be established for the respective signal 80A, 80B. As would be apparent to one of skill in the art, other raw signal features 104A, 104B can be identified.

In an analysis step, spectral density analysis 102 can be used to identify diffuse slowing. As is also shown in FIG. 8, in certain embodiments a Fast Fourier Transform 100 may be performed on the one or more signals 80A, 80B to identify additional features 104A, 104B. The Fast Fourier Transform 100 may be used alone or in combination with other analysis tools to assess spectral density. A spectral density analysis 102 may be performed on the one or more signals to determine one or more spectral density features 104A, 104B, such as low frequency density 105A, high frequency density 105B and the ratio 105C of the low and high frequency densities. These features 104A, 104B from spectral density analysis 102 may be used alone or in conjunction with the various values 104A, 104B drawn from the raw signal to predict and/or predict mortality by using a variety of optional steps and combinations.

In certain implementations, a spectral density analysis 102A, 102B can be performed on each of the one or more signals 80A, 80B, such as the EEG signals depicted, to differentiate different patient states. In certain embodiments, spectral density analysis 102A, 102B may provide values 104A, 104B including the ratio 105C of high frequency brain electrical activity to low frequency brain electrical activity. For example, the ratio of about 10 Hz signals to signals of about 2 Hz, 3 Hz, or 4 Hz can be compared to establish diffuse slowing. One or more bands or windows within the one or more signals 80A, 80B may be identified for use in the systems and methods described herein.

In certain implementations, a validation step 73 can be performed (shown in box 106). In these implementations, the spectral density 102 and/or other raw signal values 104A, 104B can be used to compare the individual readings from the partitioned signal windows (shown generally at 84A, 84B) for inclusion or rejection from the analysis. For example, in certain implementations a correction algorithm 108 can be performed. In one implementation, the error collection algorithm 108 performs a number of optional steps. In one optional step, various values 104A, 104B above or below certain pre-determined error thresholds within a given window are discarded 110. In another optional step, if the IQR and/or density ratio 105C are within a certain pre-determined proximity 112, these window signals are retained for aggregation and recombination 116, as described below. Other optional steps are possible.

In these implementations, an optional additional recombination step 116 can be performed. In the recombination step 116, windows 84A, 84B that have not been removed as a result of the validation step 73 (box 106) can be combined, such that the values 104A, 104B, 104A, 104B from those windows are aggregated as diffuse slowing threshold data 64.

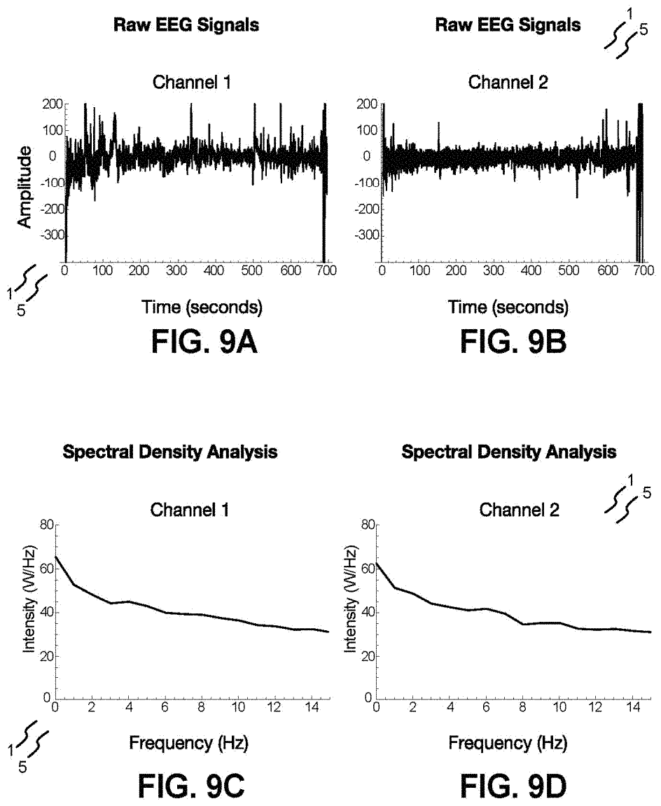

Using the diffuse slowing threshold data (shown in FIG. 6 at 64), a diffuse slowing or mortality thresholding step 74 is performed, as is further demonstrated in FIGS. 9A-D. In these implementations, the aggregated threshold data 64 can be compared against an established threshold 65 by way of the threshold module 56. In various implementations, data from other sources 66, such as electronic medical records, can also be compared against thresholds 65 to establish outputs 67, which can be graphically presented, for example, on any of the other devices and systems described herein, such as in relation to FIGS. 5A-5D. One such example is the display of the output data 67, which can include the last measured value 4, trend 6, quality 8, power 75 and other graphical representations, such as those found in FIGS. 9A-D.

As was shown in FIGS. 2A-3D, brain waves in patients that are likely to suffer mortality may be characterized by “diffuse slowing”, meaning that electrodes, preferably all electrodes, from an EEG show slowed waveforms. As noted above, brain waves may have various frequencies and/or bands of frequencies. Diffuse slowing may mean that slower waves, those with lower frequency, are seen across most, if not all, electrodes on an EEG. Emergence of slower waves as compared to the number of higher frequency waves may be an indication that a patient has or is more likely to perish. Two leads (and a ground) may be adequate for detecting diffuse slowing and, with appropriate signal processing and user interface, may require no special expertise for placement or interpretation.

In certain embodiments, and as shown in FIGS. 9A-9B, spectral density analysis 102A, 102B is performed on one or more values 104A, 104B that may be the number of high frequency waves in the one or more signals and/or the number of low frequency waves in the one or more signals. As shown in FIG. 9A, each channel 82A, 82B can be analyzed by the steps described in relation to FIG. 8A to determine the frequency of “high” and “low” frequency waves and other characteristics as output data 67. In various implementations, these representations can be used as any of the various forms of data discussed above in the process, and can be depicted on the device display 16 (shown in FIG. 4B).

In certain embodiments, the values 104A, 104B may be computed over the entirety of the one or more signals 80A, 80B as part of any of the steps described above. In certain embodiments, the values 104A, 104B may be computed over a subset of the one or more signals or a subset of time of the one or more signals. For example, if the one or more signals are five minutes in duration, the values 104A, 104B may be computed over less than five minutes, such as four minutes, three minutes, two minutes, one minute, thirty seconds, etc. Therefore, the one or more values 104A, 104B may be a feature 104A, 104B and/or values 104A, 104B over a predetermined amount of time. In certain embodiments, the one or more values 104A, 104B may be a number of high frequency waves over a period of time and/or a number of low frequency waves over a period of time. In certain embodiments, the one or more values 104A, 104B may be a ratio of a number of high frequency waves to a number of low frequency waves. In certain embodiments, the one or more values 104A, 104B may be a ratio of a number of high frequency waves over a period of time to a number of low frequency waves over a period of time.

As shown in FIGS. 8A-B and 9A-D, in various implementations of the device 10, system 1 and method 5, the ratios of high and low frequency waves for each channel can be plotted and compared at the diffuse slowing or mortality determination step (shown in FIG. 7 at 74). In these implementations, at least one of the one or more values or features or a measure based on at least one of the one or more values contained in the threshold data 64 may be compared to an established diffuse slowing threshold 65 via the module 56. In certain embodiments, a presence, absence, or likelihood of the subsequent development of mortality may be determined for a patient based on the comparison. In various implementations, the frequency is compared over time for each signal, and the results analyzed. As described further below, FIGS. 9A and 9B show the raw EEG channel signals over time for each of the respective channels. FIGS. 9C and 9D depict the resulting spectral density analysis 102 for each channel.

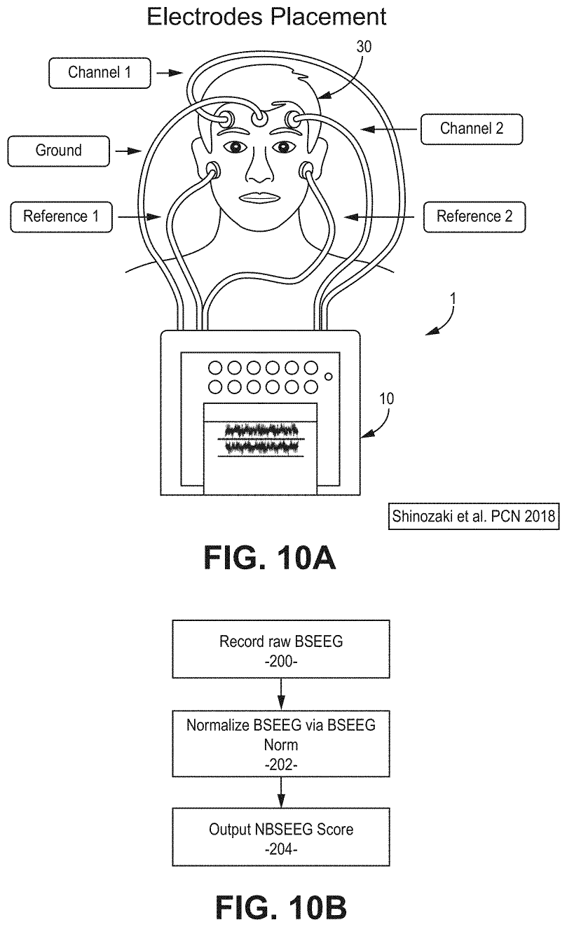

As shown in FIGS. 10A-10B, in various implementations the system 1 and method 5 are configured to execute a series of optional steps via the server/computing device 42 and/or processors 44, such as via various program modules 48, as was described in relation to FIG. 6-8. In the implementation of FIGS. 10A-10B, the system 1 is configured such that a screening device 10 is used to record raw BSEEG scores (box 200) from a subject. In these and other implementations, the outputted Normalized BSEEG (NBSEEG) score can be quantified as NBSEEG Positive (NBSEEG(+)) or NBSEEG Negative (NBSEEG(−)), which can be used to predict mortality and other negative outcomes as described herein.

In various implementations, the NBSEEG (box 202) is calculated by (the difference between the recorded raw BSEEG with a BSEEG population mean)/(the standard deviation of the BSEEG population) to output the outcome NBSEEG score (box 204) via the NBSEEG. The outcome NBSEEG can be used as categorization, such as NBSEEG Positive (NBSEEG(+)) or NBSEEG Negative (NBSEEG(−)).

Further, these outcome NBSEEG scores can be used as a continuous numbers as in the case for other vital signs such as blood pressure or body temperature. NBSEEG Positive (NBSEEG(+)) or NBSEEG Negative (NBSEEG(−)).

Experimental results are demonstrated in the accompanying examples and conclusions are given.

### Example 1: Screening Device Assessment

In this Example, the initial training set of dataset contained 186 total patient EEG samples correlated with clinical or CAM evidence of delirium. These samples represented 5 positive, 179 negative and two negative cases in which the data quality was inadequate for analysis to be performed were therefore excluded from further review.

In this Example, a 15 Hz low-pass filter was originally used, but the preliminary results indicated unequal dampening in the FFT frequency information between the positive and negative cases, therefore the low-pass filter eliminated.

During processing of the processed samples, it was observed that windows of 4 seconds were sufficient to demonstrate good results. Also, in this Example, windows containing high amplitude peaks were excluded using threshold of 500 μV for example as shown in FIGS. 9A and 9B.

FIGS. 9C and 9D depict the spectral density for the channels, wherein the intensity (in W/Hz) can be compared to each frequency (in Hz) to establish the low:high frequency ratio. It was observed that using a ratio of 3 Hz/10 Hz yielded preferable predictive results in this training set as opposed to either 2 Hz/10 Hz or 4 Hz/10 Hz ratios.

Values, features and threshold. As used herein, the terms “value” and “features” can be interchangeable, and contemplate raw and analyzed data, be it numerical, time-scale, graphical, or other. In various implementations like those described herein, the value, such as the number of high frequency waves may be compared against a threshold. Alternatively, or in addition, a ratio of two or more values may be compared against a threshold. The threshold may be a predetermined value. The threshold may be based on statistical information regarding the presence, absence, or likelihood of subsequent development of delirium, such as information from a population of individuals. In certain embodiments, the threshold may be predetermined for one or more patients. In certain embodiments, the threshold may be consistent for all patients. In certain embodiments, the threshold may be specific to one or more characteristics of the patient, such as current health, age, gender, race, medical history, other medical conditions, and the like. In certain embodiments, the threshold may be adjusted based on physiological data in a patient's electronic medical record (EMR).

In certain embodiments, the threshold may be a ratio of high frequency waves to low frequency waves. In certain embodiments, the threshold may be a ratio of high frequency waves over a period of time to low frequency waves over a period of time. Throughout the disclosure herein, the ratio is referred to as the ratio of high frequency waves to low frequency waves, but it is understood that the ratio could also be the ratio of low frequency waves to high frequency waves as long as the format of the ratio is consistent throughout out the process. For example, the comparison may be between a ratio of high frequency waves to low frequency waves or the reverse, i.e., low frequency waves/high frequency waves.

The one or more features or values may be predetermined. For example, the range for waves that are high frequency waves may be predetermined as being greater than a set value. Similarly, the range for waves that are low frequency waves may be predetermined as being less than a set value. The set values may be the same for all patients or may vary depending on specific patient characteristics.

Other features or values of the one or more signals may be extracted. For example, signal to noise ratios may also be determined for other uses. Data quality may be assessed by looking for non-physiologic frequencies of electrical activity. Data collection and/or interpretation may be limited to stopped when data quality is below an acceptable level.

Device characteristics and signal collection. The systems and methods described herein may provide a special-purpose screening device 10, system 1 and method 5 that is/are simple, convenient, and easy to use. In certain embodiments, the systems and methods may utilize electroencephalogram (EEG) technology that is simplified for an end user. The systems and methods may automatically interpret data and provide guidance to a medical professional regarding the monitoring, screening, or subsequent development of delirium by a patient. Traditionally, EEG data is visually inspected by a trained neurologist and no automation of the process is performed. In certain embodiments described herein, options may exist for interfacing with standard monitoring equipment, mobile devices, cloud technologies, and others to create an automated process.

Certain embodiments described herein may be useful in various medical areas, such as, but not limited to, intensive care, pre- and post-surgical care, geriatrics, nursing homes, emergency room and trauma care. Monitoring, screening, or predicting may improve patient care while in a hospital or other healthcare setting. Patients may also utilize personal health care devices and monitoring to allow for monitoring of their condition remotely when not in a healthcare setting. For example, personal healthcare devices may monitor patients at home or other locations outside a healthcare setting and provide monitoring, screening, or predicting of delirium. Remote sensing and/or analysis systems may interface with systems utilized by healthcare professionals.

As shown in the various implementations, the one or more sensors 12 may be placed in communication with a patient 30. In certain embodiments, the one or more sensors 12 may be one or more brain sensors, such as, but not limited to, EEG devices, such as one or more EEG leads/electrodes. For purposes of this disclosure the terms “leads” and “electrodes” are used interchangeably. The one or more signals may be EEG signals. EEG signals may include voltage fluctuations resulting from ionic current within neurons of the patient's brain. In certain embodiments, there may be a plurality of sensors. In certain embodiments, there may be two sensors, such as two EEG electrodes. The use of less than the traditional 16 or 24 electrode EEG systems may provide for reduced costs and complexities for predicting, screening, or monitoring of delirium. In various implementations, 2 or more leads or sensors are used. In certain implementations, 2, 3, 4, 5, 6, 7, 8, 9 or 10 sensors are used. In additional implementations, 11, 12, 13, 14 or 15 sensors are used. In yet further implementations, more than 15 sensors are used. In various implementations, a minimal number of easily placed EEG leads may be used—less than is demonstrated in the prior art—thereby eliminating and/or reducing the need for a skilled EEG technician and/or a sub-specialized neurologist. In various implementations, at least 1 ground lead is used, and in alternate implementations more than 1 ground lead is used, such as 2, 3, 4, 5, 6, 7, 8, 9, 10, 11, 12, 13, 14, 15, 16 or more ground leads.

In certain embodiments, the one or more sensors 12 may be non-invasive. In certain embodiments, non-invasive electrodes may be placed on the skin of a patient. In certain embodiments, there may be two skin contact points at a minimum, i.e., the sensor and the grounding sensor. There may be passive sensors in that there will be no external electrical current running through these sensors. In certain embodiments, electrically active sensors may be used. In certain embodiments, the one or more sensors may be adhesive patches with printed circuitry or a non-adhesive headset that couples the sensors to the skin. In certain embodiments, the one or more sensors may be placed on the head of a patient, such as on a forehead and/or behind one or more of the patient's ears. In certain embodiments, a minimum separation between the one or more sensors may be provided, such that the one or more sensors are not in contact with each other.

In certain embodiments, minimally invasive or invasive sensors may be used. The minimally invasive or invasive sensors may provide the one or more signals as an indication of a physiological condition, such as brain activity.

The one or more signals may be converted from analog signals to digital signals, if necessary. The conversion may be performed prior to the one or more signals being received by the processing device, at the processing device, or at a separate device. If the one or more signals are made or received as digital signals, no conversion may be necessary.

The one or more signals may be indicative of one or more brain functions of a patient. In certain embodiments, the one or more signals may provide information regarding brain wave activity of the patient. Brain waves may be measured for a patient. In certain embodiments, brain waves may be performed by EEG, which may be a recording of the electrical activity of the brain from the scalp. The recorded waveforms may reflect cortical electrical activity. In certain embodiments, signal intensity for EEG may be small, and may be measured in microvolts. Traditionally, there are several frequencies and/or bands of frequencies that may be detected using EEG. The definition of high frequency and low frequency may vary depending on various factors including, but not limited to the patient population. In certain embodiments, the definition of high frequency and low frequency may be consistent across patient populations. In certain embodiments, low frequency waves may be waves with less than approximately 7.5 Hz, less than, approximately 7.0 Hz, less than approximately 6.5 Hz, less than approximately 5.5 Hz, less than approximately 5 Hz, less than approximately 4.5 Hz, less than approximately 4.0 Hz, less than approximately 3.5 Hz, or less than approximately 3.0 Hz. In certain embodiments, high frequency waves may be waves with more than approximately 7.5 Hz, more than approximately 8.0 Hz, more than approximately 8.5 Hz, more than approximately 9.0 Hz, more than approximately 9.5 Hz, more than approximately 10.0 Hz, more than approximately 10.5 Hz, more than approximately 11.0 Hz, more than approximately 11.5 Hz, more than approximately 12.0 Hz, more than approximately 12.5 Hz, more than approximately 13.0 Hz, or more than approximately 14.0 Hz.

In certain embodiments, the one or more signals may be real-time or near real-time streams of data. In certain embodiments, the one or more signals may be measured and/or stored for a period of time before processing and/or analysis.

Although not required, the systems and methods are described in the general context of software and/or computer program instructions executed by one or more computing devices that can take the form of traditional servers/desktops/laptops; mobile devices, such as a Smartphone or tablet; wearable devices, medical devices, other healthcare systems, etc. Computing devices may include one or more processors coupled to data storage for computer program modules and data. Key technologies may include, but are not limited to, multi-industry standards of Microsoft and Linux/Unix based Operating Systems; databases such as SQL Server, Oracle, NOSQL, and DB2; Business Analytic/Intelligence tools such as SPSS, Cognos, SAS, etc.; development tools such as Java, .NET Framework (VB.NET, ASP.NET, AJAX.NET, etc.); and other e-Commerce products, computer languages, and development tools. Such program modules may include computer program instructions such as routines, programs, objects, components, etc., for execution by the one or more processors to perform particular tasks, utilize data, data structures, and/or implement particular abstract data types. While the systems, methods, and apparatus are described in the foregoing context, acts and operations described hereinafter may also be implemented in hardware.

### Example 2: NBSEEG, Delrium & Mortality

Methods: This is a prospective study to measure bispectral EEG (“BSEEG”) from the elderly inpatients to assess their outcomes. A normalized BSEEG (“NBSEEG”) score was defined based on the distribution of 2938 BSEEG recordings from the 428 subjects, who were assessed for delirium; primary outcomes measured were hospital length of stay (“LOS”), discharge disposition, and mortality.

Results: 274 patients had NBSEEG scores data available for analysis. Delirium and NBSEEG score had a significant association (P<0.001). Higher NBSEEG scores were significantly correlated with LOS (P<0.001) as well as with discharge not to home (P<0.01). Hazard ratio for survival controlling for age, gender, Charlson Comorbidity Index and delirium status was 1.35 (95% confidence interval=1.04 to 1.76, P=0.025).

Described herein is an efficient and reliable device that provides an objective measurement of brain function status. The NBSEEG score is significantly associated with pertinent clinical outcomes of mortality, hospital length of stay, and discharge disposition. The NBSEEG score actually better predicts mortality than clinical delirium status. Use of the device and system described herein allow for identification of a previously unrecognized sub-population of patients without clinical features of delirium who are at increased mortality risk.

Electrophysiological signals characteristic of delirium are often reported as “diffuse slowing.” The term implies that brain waves across most channels are of a reduced frequency. The emergence of low-frequency waves indicates potential occurrence of delirium. The fact that all channels are able to detect the same reduction in frequency suggests only a small number of channels would be sufficient to obtain the relevant data. BSEEG utilizes only two channels, and when combined with appropriate signal analysis algorithms, may be easily applied by non-experts, thus greatly facilitating its use as a screening tool. Due to its objective nature, inter-rater reliability does not affect BSEEG and it can be more strongly correlated with patient outcomes. The below example illustrates a study of whether BSEEG values and NBSEEG scores can predict patient outcomes, including mortality.

Methods.

Study Design and Oversight. This is to test the usefulness of BSEEG approach on patient care, the association of NBSEEG scores from this algorithm and patient outcomes were investigated.

Variables and Data Sources. For measurement of clinical symptoms of delirium, the CAM-ICU, the DRS-R-98, and Delirium Observation Screening Score (“DOSS”) were used. For the assessment of baseline cognitive function the Montreal Cognitive Assessment (MoCA) was used. CAM-ICU and DRS-R-98 were administered to each subject twice daily, unless the subject declined that instance of assessment. DOSS was tabulated by clinical nursing staff during their routine care and was obtained from review of the medical record. Delirium was defined based on any questionnaire screening positive, i.e. CAM-ICU positive, DRS-R-98≥18, DOSS>2, or clinical documentation of altered mental status or confusion consistent with delirium from the medical record. Each case was reviewed by a weekly research meeting led by a board-certified consult-liaison psychiatrist.

BSEEG Data Collection. A hand-held, two-channel EEG device was used for brain wave recording. Raw BSEEG vales were collected twice daily, unless the subject declined that instance of assessment. One electrode on the center of forehead as a ground, two electrodes were placed on the left and right sides of the forehead, and two electrodes were placed on both sides of the earlobe as references to obtain raw BSEEG values for 10 min, as shown in FIG. 10. The 10 minute duration was chosen as an amount of time that allowed for collection of an adequate amount of EEG data without sacrificing efficiency and throughput—essential features of a screening test. The recording was obtained while the patient was at their highest level of consciousness such that the patient was as awake and alert as their clinical status allowed. Patients were instructed to keep their eyes closed, jaw relaxed, and remain quiet and still as much as possible. The obtained raw BSEEG value data was converted into spectral density plots, and the signal-processing algorithm was used to produce a raw BSEEG value or values.

Spectral Density Analysis and NBSEEG Score. Raw EEG signal from each channel was subjected to power spectral density analysis to determine relative presence of “high” and “low” frequency components. Through an iterative approach, a score reflecting the relative presence of high and low frequency activity was developed. From 2938 recordings of raw BSEEG values from all 428 study participants, a mean value and standard deviation (SD) was calculated, shown in FIG. 11. The NBSEEG score was defined as the number of SD from the study population mean.

Outcome Measures. Three patient outcomes were tracked and measured as follows: 1) hospital LOS; 2) discharge not to home, which included death during hospitalization; and 3) mortality at the time of study conclusion. LOS, discharge outcome, and mortality status were obtained through each subject's hospital record. Mortality was also assessed by a follow-up phone call interview and obituary record.

Statistical Methods and Analysis. Regression analyses were used to illustrate how the proposed NBSEEG score is associated with clinical delirium and patient outcomes such as hospital LOS, discharge not to home, and mortality. Specifically, logistic regression was conducted by treating delirium and discharge not to home as binary response variables, respectively, while linear regression was used to evaluate the relationship between hospital LOS and NBSEEG score. In addition, the hazard ratio for mortality was computed through Cox proportional hazards regression analyses. Age, gender, and severity of illness were controlled in regression analyses. The association between mortality and NBSEEG scores was further illustrated by comparing two non-parametric survival functions for NBSEEG-positive and NBSEEG-negative groups. The survival function is a series of the Kaplan-Meier estimators obtained from the number of deaths and the total individuals at risk at the time. The log-rank test was conducted to determine whether the two survival functions differ. Two-sided P-values of 0.05 or less were considered to indicate statistical significance. All analyses were performed with R software, version 3.4.3.

Results.

Participants, Descriptive Data and Outcome Data. 428 patients were enrolled in the study. 337 out of 428 patients were 55 years old or older and 274 out of 337 had NBSEEG scores available for analysis. In the group 55 years old or older, 37.2% of patients were categorized as delirious by questionnaire screening or clinical documentation. The study population was also independently divided into two groups, NBSEEG-positive, with higher NBSEEG scores, indicative of more low-frequency components in their brain waves, and NBSEEG-negative, with lower NBSEEG scores, indicative of less low-frequency components in their brain waves, based on a threshold to differentiate patient outcomes as described in the following section, as shown in FIG. 12. Otherwise, these cohorts were balanced with respect to overall baseline characteristics shown in Table 1.

Association between NBSEEG Score and Clinical Delirium. Data from 274 subjects were analyzed to establish association between NBSEEG score and clinical delirium. Logistic regression showed significant association between delirium category and NBSEEG score (P=6.39×10⁻⁶, unadjusted; P=1.22×10⁻⁵, adjusted for age, gender, and CCI).

NBSEEG Score and Patient Outcomes: To test the usefulness of the NBSEEG score in predicting patient outcomes, we used outcome data available from 274 subjects who were 55 years old or older to investigate the association of NBSEEG scores obtained at the time of study enrollment with patient outcomes commonly affected by delirium. Specifically, assessed were hospital LOS, discharge disposition, and mortality.

First, LOS and NBSEEG scores were significantly associated (P=0.00099, unadjusted; P=0.0014, adjusted for age, gender and CCI). A higher NBSEEG score coincides with an increase in a patient's LOS.

Second, the discharge outcome and NBSEEG score were compared. When NBSEEG was compared between those who were discharged to their home and those discharged not to home, including death during hospitalization, a higher NBSEEG score was significantly associated with discharge not to home (P=0.0038, unadjusted; P=0.0090, adjusted for age, gender, and CCI).

Third, subject mortality was analyzed controlling for age, gender, and CCI, the hazard ratio based on 1 SD change of NBSEEG score was 1.44 (1.12 to 1.84, P=0.004). Even after controlling for clinical delirium status in addition to age, gender and CCI, the HR based on NBSEEG score remained significant at 1.35 (95% confidence interval=1.04 to 1.76, P=0.025).

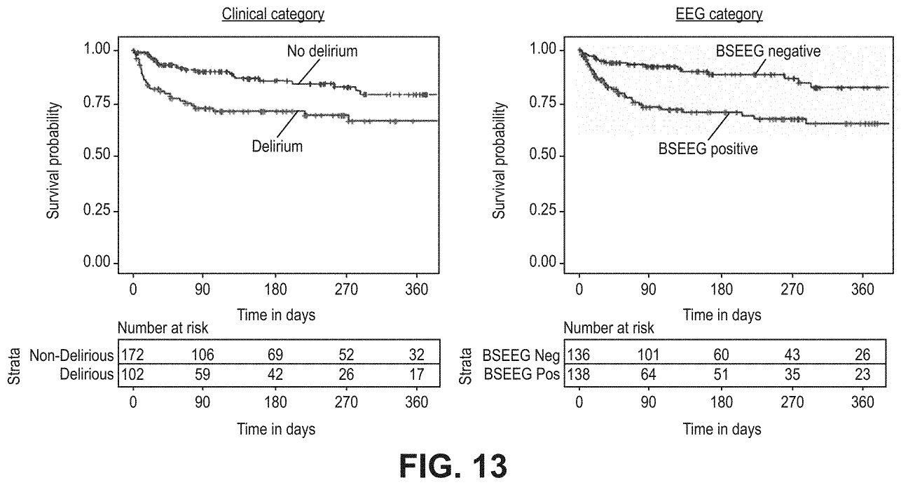

Besides mathematical association, NBSEEG score was analyzed as a potentially useful measure to assess risk for poor patient outcomes. The study population was divided into a NBSEEG-positive group and a NBSEEG-negative group, as mentioned above. Then, the data was assessed to determine if there was a correlation between groups based on NBSEEG scores and all-cause mortality at the end of our study period in patients in our dataset. First overall survival rates were assessed among study participants to confirm if the clinical categorization of delirium is valid enough to replicate well established association between delirium and higher mortality. Results showed differences in mortality between those with and without clinical delirium (P=0.0038) FIG. 13. Second, a group difference was tested based on a NBSEEG cut-off score and confirmed that the NBSEEG-positive group showed worse survival compared to the NBSEEG-negative group (P=0.0032) (FIG. 13). This categorization also differentiated other outcomes including LOS and discharge disposition significantly (Table 2).

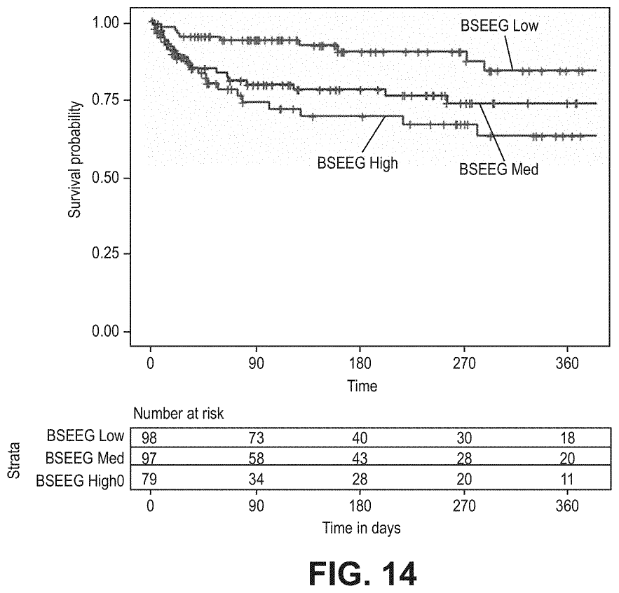

NBSEEG score not only measures the presence of delirium, but represents delirium severity. Subjects were divided into three groups based on NBSEEG score: NBSEEG high, NBSEEG intermediate, and NBSEEG low. The survival curve showed a “dose-dependent” relationship of increasing mortality with increasing NBSEEG score (FIG. 14, P=0.005), suggesting a strong relationship between NBSEEG score and mortality.

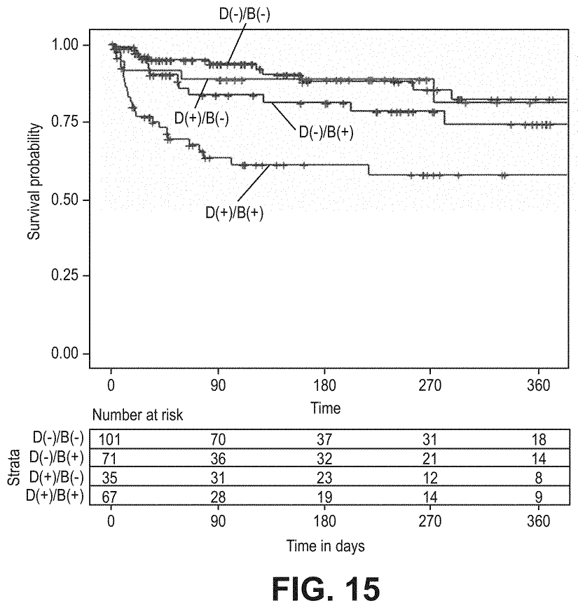

The cohort was divided into four groups based on clinical delirium diagnosis and BSEEG. Clinically delirious subjects with positive NBSEEG scores showed the highest mortality. In contrast, those patients categorized as clinically delirious but with a negative NBSEEG score had lower mortality, similar to that of non-delirious subjects with negative NBSEEG scores. Moreover, those thought to be non-delirious subjects based on results of clinical assessment but with positive NBSEEG scores had a higher mortality, even compared to those patients with clinical delirium but with a negative NBSEEG score (FIG. 15).

Discussion—Key results and Interpretation: NBSEEG scores were significantly associated with the clinical presence of delirium, even after controlling for age, gender, and CCI. More importantly, NBSEEG scores were strongly associated with patient outcomes, including hospital LOS, discharge disposition, and mortality among hospitalized patients. Importantly, this association was based on a NBSEEG score obtained at the time of enrollment, often within 24 hours after admission. These results suggest that a single NBSEEG score obtained at the beginning of hospitalization can predict patient outcomes. This result also indicates that among patients who cannot be clinically identified as delirious, a subset are at high risk of death that is distinguishable by differences in brain wave activity as detected by NBSEEG. This state can be categorized as subclinical brain failure (“SBF”). Thus, identification of this population with the NBSEEG method could lead to early intervention and potentially improved survival rates.

The data disclosed herein demonstrates the usefulness of the NBSEEG score in differentiating delirium cases from non-delirious patients and in predicting patient outcomes, such as hospital LOS, discharge disposition, and mortality, among elderly hospitalized patients.

Such NBSEEG-based biomarkers may enable early intervention and improve the current practice of medicine and surgery for patients at risk of delirium. For example, NBSEEG analysis may be an important factor in the decision to perform elective surgery or be used for heightened monitoring after surgery. When high-risk patients are identified through NBSEEG analysis, it is then possible to direct hospital resources more efficiently and effectively compared to the current standard of care.

NBSEEG monitoring may also be applicable in additional settings such as the primary care clinic, emergency department, and in nursing home or home-care settings. Delirium is particularly dangerous when patients experience it outside of hospitals because of the lack of recognition and resources to manage it. The simple, noninvasive nature of this test makes it ideal for routine screening. Further the NBSEEG test described herein can be used as a monitoring tool to assess the risk of mortality in appropriate populations. For example, as the aging population is expanding rapidly, NBSEEG can be implemented as an efficient modality for mortality risk screening.

Limitations. Electrode placement on the forehead was used as a user-friendly screening method. Of course other lead placement conformations may be possible.

In some implementations the devices and methods described herein can be used to explore and evaluate the effect of various treatments, such as ramelteon and suvorexant, to determine the impact of certain treatments/medications on NBSEEG score and outcomes. As such, the disclosed devices and methods may be used to provide and explore better treatments for delirium and, ultimately, improve patient outcomes.

Implications for Practice. A noninvasive, point-of-care EEG collection combined with NBSEEG scoring is able to predict adverse patient outcomes, including mortality. Importantly, certain patient populations can be identified where the patients cannot be identified by current clinical assessments, but are at high risk for mortality.

### Example 3

This example evaluates the use of two channel frontal EEG activity to quantitatively characterize delirium and predict outcomes including fall risk and mortality.

Methods.

Frontal EEG activity (Fp1 and Fp2 EEG locations) was collected from patients after admission or at the time of an emergency room visit. Subjects were assessed for the clinical presence of delirium and the primary outcomes measured were delirium diagnosis, discharge disposition, mortality, and fall history. EEG features (band powers and different combinations of low to high frequency activity) were calculated for both channels and averaged. K-nearest neighbors, logistic regression, support vector machine (SVM), kernelized SVM, and neural network approaches were used to assess the ability of EEG features to predict delirium status, survival, and falls with 5-fold cross-validation.

Results.

EEG features and outcome data for 274 inpatients were available for analysis. The top 9 EEG-derived predictive features were selected using Random Forest. Of all the classification methods, kernelized SVM yielded the highest prediction accuracies of 69%, 81%, 89% for delirium status, mortality, and falls respectively. Frontal EEG may be used in objectively measuring delirium from a variation of clinical causes, and can predict pertinent clinical outcomes including fall risk and mortality.

Placing only two channels—BSEEG—on the head allows for non-experts to apply the device, thereby removing the necessity of specialized neurologists and technicians and permitting mass adaptation of the technology. In this example, the disclosed BSEEG method along with a point-of-care technology was used to predict patient outcomes (delirium diagnosis, mortality, fall risk, and discharge status). Specifically, whether power spectral density analysis from limited forehead EEG leads can predict patient outcomes.

Methods.

Questionnaire instruments and delirium definition. For measurement of clinical symptoms of delirium, the CAM-ICU, DRS-R-98, and DOSS were used. For the assessment of baseline cognitive function the MoCA was used. Delirium was defined based on questionnaire screening positive, such as CAM-ICU positive, DRS-R-98>18, DOSS>2, or documentation of altered mental status or confusion consistent with delirium from medical record. Each case was reviewed by a weekly research meeting led by the board certified psychosomatic medicine psychiatrist.

BSEEG Data Collection and Handling Protocol. A hand-held, two-channel EEG device was used for brain wave recording. One electrode was placed on the center of forehead as ground, two electrodes were placed on the left and right sides of the forehead, and two electrodes were placed on both sides of the earlobe as references to obtain BSEEG signals for 10 minutes. The obtained BSEEG data was converted into spectral density plots and the signal-processing algorithm was performed to extract EEG features.

EEG signal processing, analysis, and interpretation. Recorded EEG data were exported in European Data Format for further analysis. Each channel of EEG data was extracted and subsequently divided into 4-s windows which were then filtered for excessive noise. Partitioned windows with interference were removed from further analysis. The power spectral density (PSD) of the remaining windows were obtained via fast Fourier transformation, and aggregated as the median of all remaining windows. BSEEG features (band powers and different combinations of low to high frequency activity) were calculated for both channels and averaged. Various PSD ratios (PSDR) of low-to-high frequency activities were used to obtain a features for further analysis at baseline admission.

Outcomes. Patient outcomes were measured as follows: 1) delirium diagnosis, 2) survival, 3) discharge not to home including death during hospitalization. All outcomes were obtained through patient hospital record. Mortality was also assessed through follow up phone call interview and obituary record. An end-point classification was determined by the research members, who were unaware of the BSEEG features.

Predictive Model Using Random Forrest: The predictive power of BSEEG features was assessed using Random Forrest (RF) with the Boruta algorithm, which can predict disease status based on an ensemble of decision trees. RF was used to build a predictive model based on the EEG profile using all EEG features as the input. The relative importance of each EEG feature in the predictive model was assessed using mean decreasing accuracy and Gini coefficient.

Classification Algorithms: After establishing association between delirium status and EEG features, classification analyses were used to test whether the development of poor outcomes, such as mortality and fall risk, are associated with EEG signals. A variety of machine learning (ML) algorithms were applied to the EEG dataset for different types of classification tasks. The classification models achieved through learning were directly used for prediction of patient outcomes such as delirium, mortality, fall, and discharge. In the machine learning hierarchy, classification tasks fall under supervised learning tasks, which means that, unlike unsupervised learning tasks, there is feedback available to the learning system. That feedback is also referred to as gold standard, training data, example data, or labeled data.

K-Nearest Neighbors. The k-Nearest Neighbors algorithm (k-NNs) stores the training data. When a new data point that comes in, the k-NNs finds the points in the training dataset that are closest, or nearest, to the new data point. Here, k is the number of closest neighbors to consider. The k-NNs can then make a prediction using a majority vote among the k nearest neighbors. The only input parameter that the k-NN algorithm takes for learning is k. In our model, we adjusted the value of k from 1 to 10 to capture the k that gave the best prediction accuracy on the test dataset. The k-NNs algorithm is very easy to understand and often gives reasonable performance without significant adjustments. On the other hand, it is slow in prediction for large training datasets, as it needs to calculate in real time the distances between the new data point and all of the data points in the training dataset. It also does not perform well on datasets with many features or sparse datasets. For these reasons, the k-NNs algorithm is not often used in practice and instead serves as a good baseline method to try before considering advanced techniques.

Logistic Regression. The Logistic Regression algorithm is based on the Linear Regression algorithm that makes a prediction of a target variable using a linear function of input feature variables. The difference between the two algorithms is that the Linear Regression algorithm make a prediction about a continuous number, while the Logistic Regression algorithm about a predefined class label, so that it can be used for classification tasks. In order to make a prediction about a class label, the entire linear function is put into another function, called a sigmoid function, which ranges between 0 and 1. If the sigmoid function is larger than 0.5, it predicts the class as +1; if smaller than 0.5, it predicts as −1. The Logistic Regression algorithm is a binary classification algorithm that returns +1 and −1. A number of techniques such as one-vs.-all, or one-vs.-rest, have been proposed to extend a binary classification algorithm to a multiclass classification algorithm that can be used for classification tasks handling multiple classes. Linear models are fast to train and to predict. They scale to very large datasets and work well with sparse data. They are relatively easy to understand how a prediction is made using linear functions. On the other hand, linear models, as its name indicates, are based on a strong assumption that the target variable can be predicted by a linear combination of feature variables, which may be too weak to apply to real world problems.

Support Vector Machines. The Support Vector Machines algorithm, or SVMs, is based on the large margin intuition. In other words, it tries to find the maximum-margin strict line, plane, or hyperplane that represents the largest separation, or margin, between two classes. Typically, only a subset of the data points matters in defining the decision boundary, especially the ones that lie on the border between the classes, which are called support vectors. In order to make a prediction for a new data point, the distance to each of the support vectors is measured, and the classification decision is made based on the distances to the support vector and the weights of the support vector that were learned during training. The SVMs algorithm works well with high-dimensional data, which means it can draw complex decision boundaries. In this case, the distance between data points can be measured by the Gaussian kernel. The SVMs algorithm that uses the Gaussian kernel function is called the Kernelized SVMs algorithm. The two parameters that we adjusted for learning are the penalty parameter of the error term for regularization and the kernel coefficient, or gamma. The SVMs algorithm perform very well on a variety of datasets, which is the reason why it is known as one of the most commonly-used classification algorithms. It allows for complex decision boundaries, as described above, even if the dataset has only a few features. It also works well on low-dimensional data with few features and high-dimensional data with many features. On the other hand, it is very sensitive to the scaling of data and the settings of parameters. It is sometimes hard to understand why a particular decision was made by the algorithm.

Neural Networks. The Neural Networks algorithm was inspired by the real biological neural networks that constitute animal brains. The algorithm is basically the generalizations of linear models that perform multiple stages of processing to come to a decision. For example, a Logistic Regression model can be represented as a two-layer Neural Networks that consists of an input layer with input feature nodes and an output layer with a target node. Then, a new hidden layer with several hidden nodes can be added between the input and output layers to make the model more complex. Various hidden layers and hidden nodes can be added to make the model more complex. All initial weights are set randomly, and thus this random initialization can affect the model that is learned. We adjusted the value of random state for the random initialization of weights from 0 to 10 to capture the random state that gave the best prediction accuracy on the test dataset. The Neural Networks algorithm is able to capture information contained in large amounts of data and build very complex models. It often outperforms other machine learning algorithms, given sufficient computation time, data, and careful tuning of parameters. On the other hand, it often takes long time to train. It also requires careful preprocessing of data and tuning of parameters.

Results.

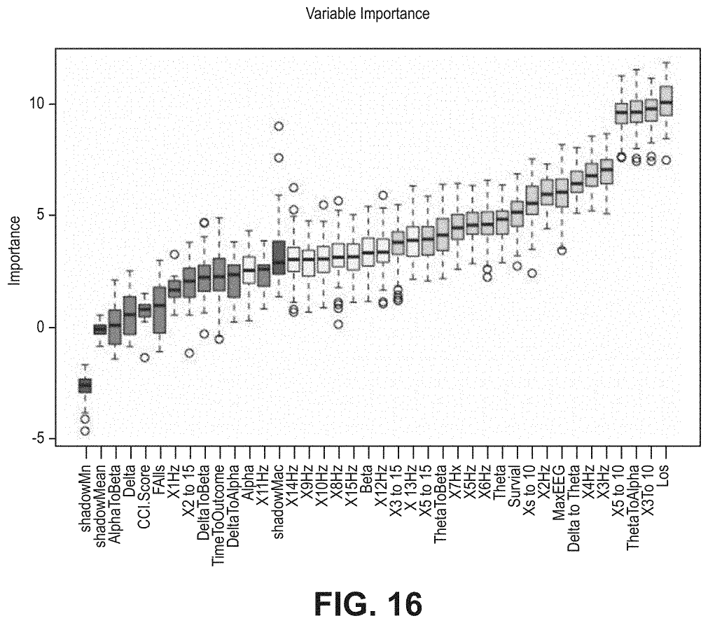

The Boruta algorithm was used to select significant BSEEG features, and 9 genera were confirmed for their importance in prediction, shown in FIG. 16. The prediction model confirmed that certain BSEEG features, particularly the 3 to 10 Hz PSD ratio, theta to alpha power ratio, and 5 to 10 Hz PSD ratio were predictive of the delirium state.

Outcomes of delirium diagnosis, survival, falls, and discharge not to home including death during hospitalization, were predicted using KNN, logistic regression, SVM, Kernelized SVM, and neural network algorithms. The results of all analyses are presented in Tables 3-6.

Discussion

The results show the utility of a simplified, portable, automated EEG with bispectral density analysis (BSEEG method) for predicting patient outcomes. Compared to traditional EEG, which requires >20 leads placed all over the head of patients by a trained EEG technician, the system requires only a few leads placed on the forehead, thus requiring minimal training. Screening can be achieved in minimal time (i.e. minutes), and extended monitoring can also be performed, even dynamically, with recordings of longer duration. This is a significant advantage compared to a traditional EEG reading interpreted by specialists, which introduces significant delays. BSEEG is also an improvement over numerous screening methods currently used in practice, such as questionnaire-style methods, which are prone to subjective variation by examiners, as well as mental status exams, which requires extensive training and prolonged time to conduct.

Continuous monitoring with BSEEG can categorize patients into three or more different levels of moralities. A regular EEG as read by a neurologist is only capable of a dichotomous classification of patients, either diffuse slowing or normal. In fact, BSEEG is capable of predicting mortality as well as a traditional EEG.

BSEEG is an improvement in that it requires fewer electrodes and does not require interpretation by a specialized neurologist. In various implementations, BSEEG can provide a continuous measurement, thereby providing more comprehensive and additional information on the risk of mortality than can be obtained from a traditional EEG and a neurologist.

The BSEEG devices and methods described herein may further be used to predict delirium-relevant patient outcomes. In clinical practice, this method may be used as an additional biomarker to predict patient outcomes. Electrodes may be placed on the forehead, while other lead placement conformations are also possible. In various implementations discussed herein, the EEG-derived features were just as important as traditional clinical metrics such as hospital length of stay in predicting delirium status (FIG. 16).

BSEEG may be useful to differentiate delirium cases versus normal subjects, and also to predict patient outcomes, including mortality, fall risk, and discharge outcomes among elderly hospitalized patients. BSEEG may be used not only for patients with obvious mental status change, but also for a broader cohort of patients.

BSEEG can enable early intervention and prevention of delirium-related outcomes, and can improve the current practice of medicine for patients as risk of delirium. For instance, when high risk patients are identified through BSEEG analysis, it is then possible to direct hospital resources more efficiently and effectively compares to current standard of care. BSEEG monitoring may also be applicable in additional settings such as the primary care clinic, emergency department, and in nursing home or home-care settings. Delirium is particularly dangerous when patients experience it outside of hospitals because they do not have medical attention available on site. The devices, systems, and methods discussed herein may be used for routine screening and monitoring.

### Example 4

Prediction of one month all-cause-mortality by NBSEEG. A NBSEEG score can detect delirium, and separately predict mortality in elderly inpatients as soon as in 30 days or less. The NBSEEG score can predict mortality among dementia patients.

Results: The mortality in 180 days in the NBSEEG positive group was higher than those of NBSEEG negative group both in the replication cohort (N=228) and the combined cohort (N=502). Their mortality showed dose-dependent increase in both cohorts. The mortality in 30 days in the NBSEEG positive group was significantly higher than those of the negative (relative risk=3.65; 95% CI, 1.73 to 7.69; P<0.001). When the dementia patients showed NBSEEG positive, their mortality was significantly higher than those with dementia but with NBSEEG negative in 60 days (relative risk=3.00; 95% CI, 1.17 to 7.70; P=0.025) and 90 days (relative risk=3.80; 95% CI, 1.52 to 9.48; P=0.002).

Delirium was screened by using the following questionnaire; the CAM for the Intensive Care Unit (CAM-ICU), the Delirium Rating Scale-Revised-98 (DRS-R-98), and the Delirium Observation Screening Scale (DOSS). Delirium status was defined according to the results of the following screenings; CAM-ICU positive, DRS-R-98 score ≥19, or DOSS score ≥3. Baseline cognitive function was measured by using the Montreal Cognitive Assessment (MoCA). Dementia was recorded based on a chart review. Delirium and dementia status was finally determined by a board-certified consultation-liaison psychiatrist with the results of the measures and detailed chart review.

NBSEEG data were collected by using a portable EEG device, such as is shown in FIG. 10A.

All statistical analyses were conducted using R. A t-test was conducted to compare continuous data between case and control for the delirium and dementia, and positive and negative for the NBSEEG score. A log rank test was conducted to compare two survival functions in 180 days. Moreover, mortality of both NBSEEG positive and negative groups at the time of 30 days was compared to test how soon NBSEEG can differentiate mortality risk. In addition, relative risk of the mortality in 30 days was calculated between the NBSEEG positive and negative groups. Cox proportional hazards regression analysis was conducted to calculate the hazard ratio adjusting age, sex, and the Charlson Comorbidity Index (CCI). A p value of less than 0.05 was determined as statistical significant.

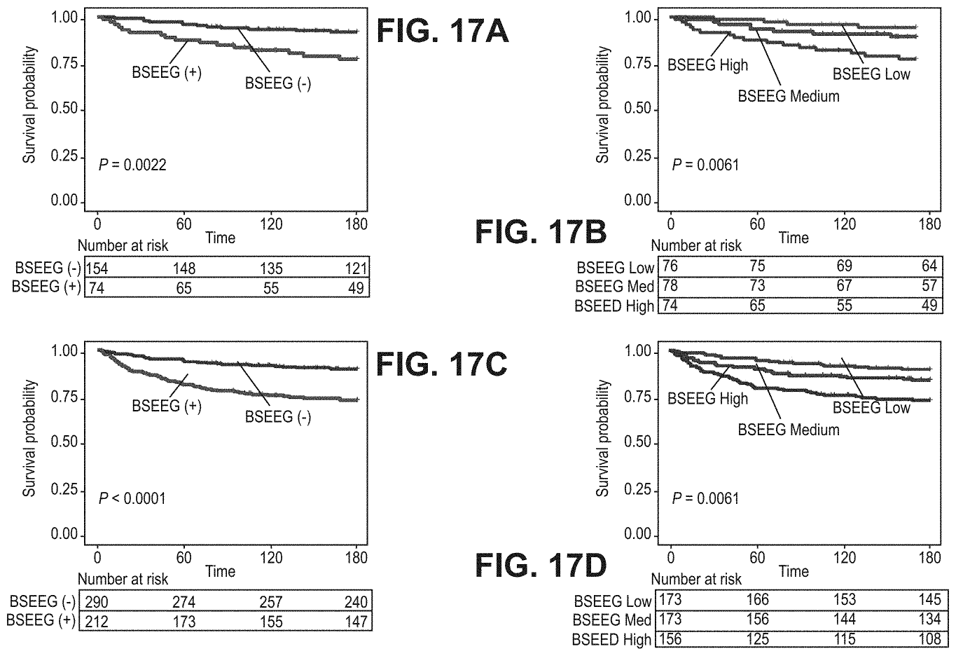

Results—Replication for utility of NBSEEG in prediction of mortality. Analysis of data from 228 subjects (replication cohort) confirmed the utility of NBSEEG in prediction of mortality. The demographic characteristics of them were shown in Table 7. Age and CCI were significantly higher in patients with delirium, dementia, and NBSEEG positive groups, compared to each control groups (Table 7). The proportion of female was significantly higher in patients with dementia compared to control group (Table 7). The unadjusted mortality in 180 days in NBSEEG positive group was higher than those of NBSEEG negative group (FIG. 17A). When the patients were divided into 3 categories to become approximately equal sample sizes with NBSEEG low, medium, and high based on the NBSEEG scores, their mortality showed dose-dependent increase based on the NBSEEG categories (FIG. 17B). According to the result of the Cox proportional hazard model adjusted with age, sex, CCI, and delirium, the NBSEEG showed significant predictive factor for mortality in 180 days (95% CI, 1.33 to 6.00; p=0.007) (Table 8). In addition, age and CCI were significant predictors for mortality (Table 8).

The demographic characteristics of 502 analyzed subjects are shown in Table 9. Age and CCI were significantly higher in patients with delirium, dementia, and NBSEEG positive groups, compared to each control groups (Table 9). The unadjusted mortality in 180 days in NBSEEG positive group was higher than those of NBSEEG negative group (FIG. 17C). Moreover, when the patients were divided into 3 categories to become approximately equal sample sizes with NBSEEG low, medium, and high based on the NBSEEG scores, their mortality showed score dependent increase based on the NBSEEG categories (FIG. 17D). According to the result of the Cox proportional hazard model adjusted with age, sex, CCI, and delirium, the NBSEEG showed significant predictive factor for mortality in 180 days (95% CI, 1.55 to 3.82; p<0.001) (Table 10). Age, delirium status and CCI were significant predictors for mortality (Table 10).

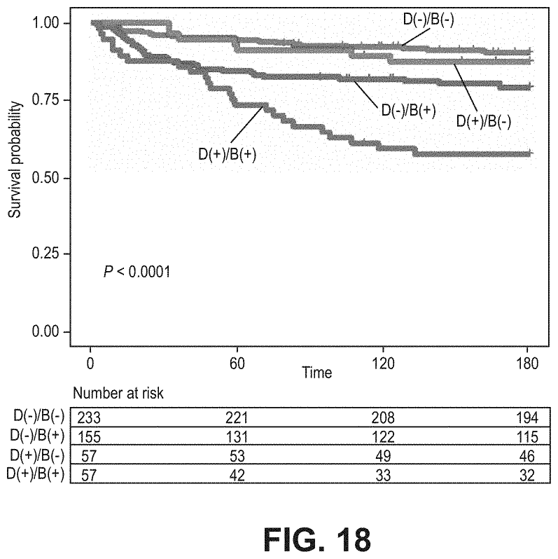

Utility of NBSEEG in predicting mortality among patients with and without dementia: The 502 subjects were analyzed to test the utility of NBSEEG for predicting mortality in patients with dementia. When the patients with dementia showed NBSEEG positive, their mortality was higher than those with dementia but with NBSEEG negative (FIG. 18). When dementia was added as a covariate in the Cox proportional hazard model, the BSEEG still showed significant predictive factor for mortality in 180 days (95% CI, 1.55 to 3.82; P<0.001) (Table 11). Similarly, age, delirium, and CCI were significant predictors for mortality (Table 11).

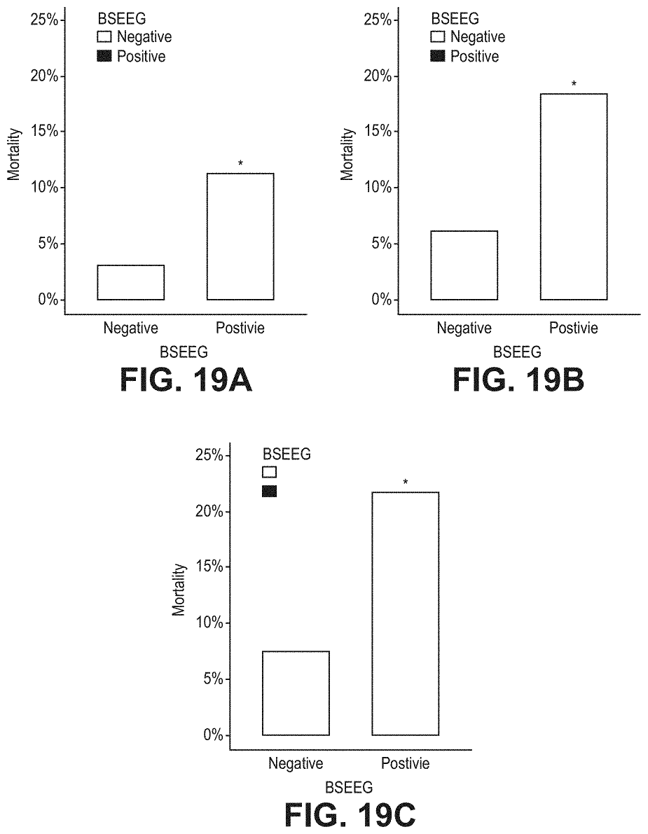

Utility of NBSEEG in predicting short-term mortality: The mortality in 30, 60, and 90 days were compared to test how soon NBSEEG can differentiate mortality risk among the total 502 subjects (discovery and replication cohorts). The mortality in NBSEEG positive group in 30 days was significantly higher than those of NBSEEG negative group (relative risk=3.65; 95% CI, 1.73 to 7.69; p<0.001) (FIG. 19A). Similarly, the mortality in NBSEEG positive group in 60 days was significantly higher than those of NBSEEG negative group (relative risk=2.96; 95% CI, 1.74 to 5.03; p<0.001) (FIG. 19B), as well as the mortality in NBSEEG positive group in 90 days compared to the negative group; (relative risk=2.86; 95% CI, 1.78 to 4.60; p<0.001) (FIG. 19C).

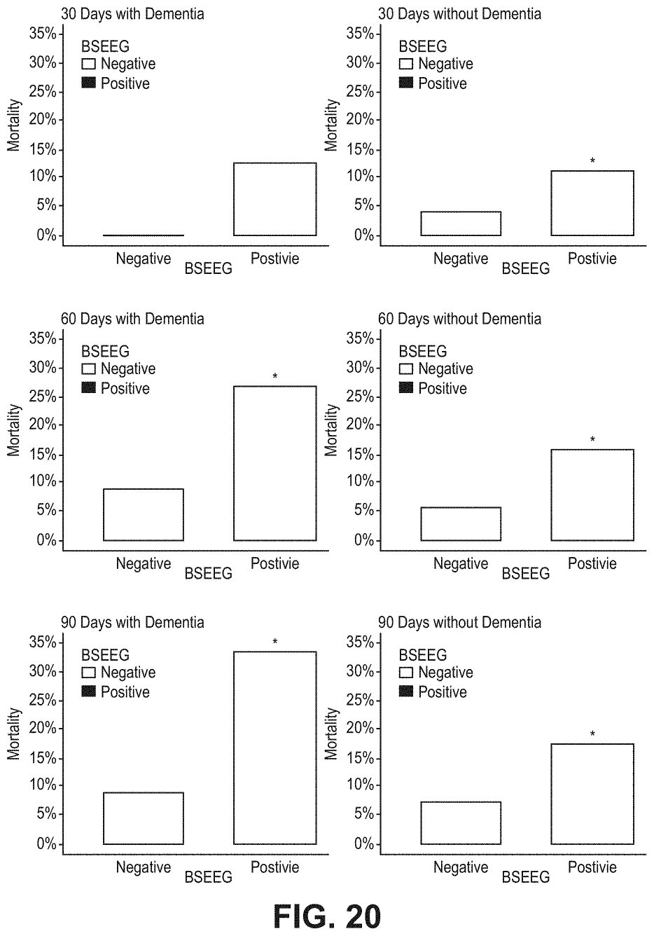

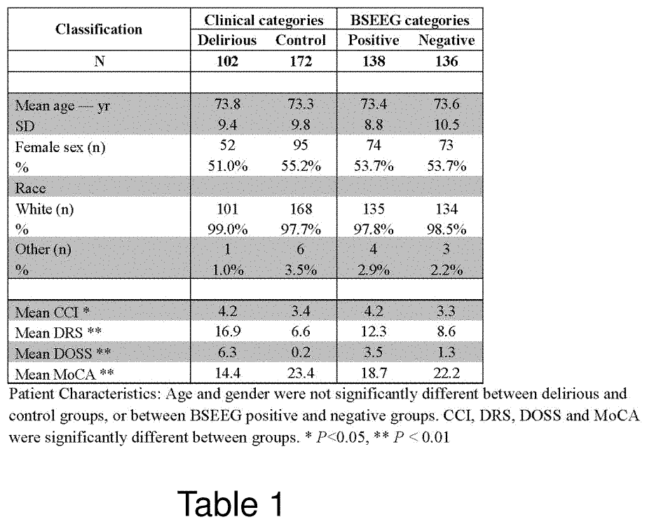

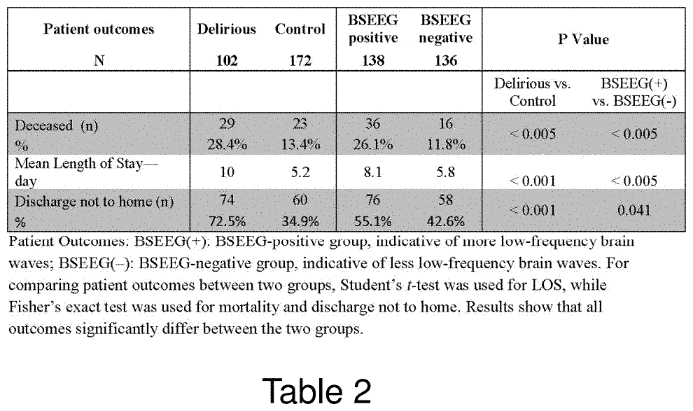

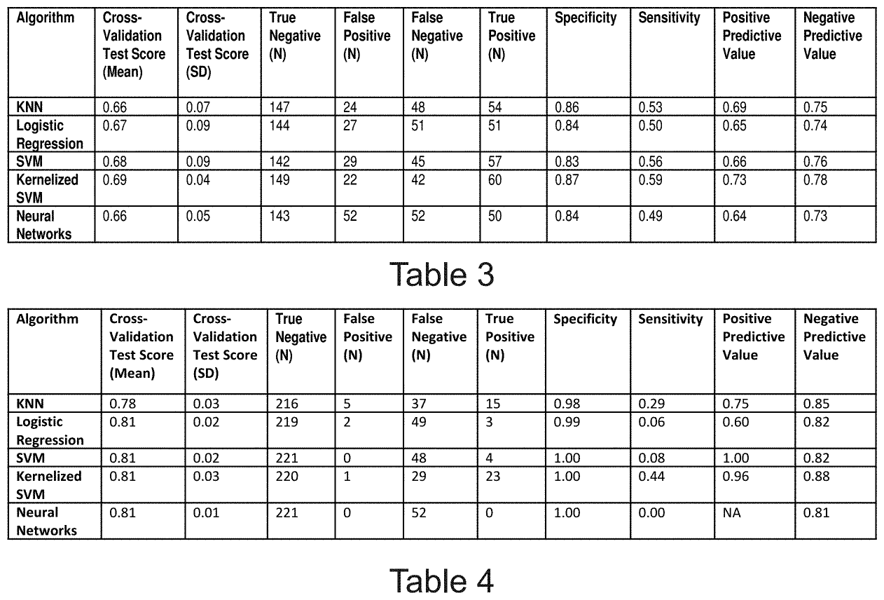

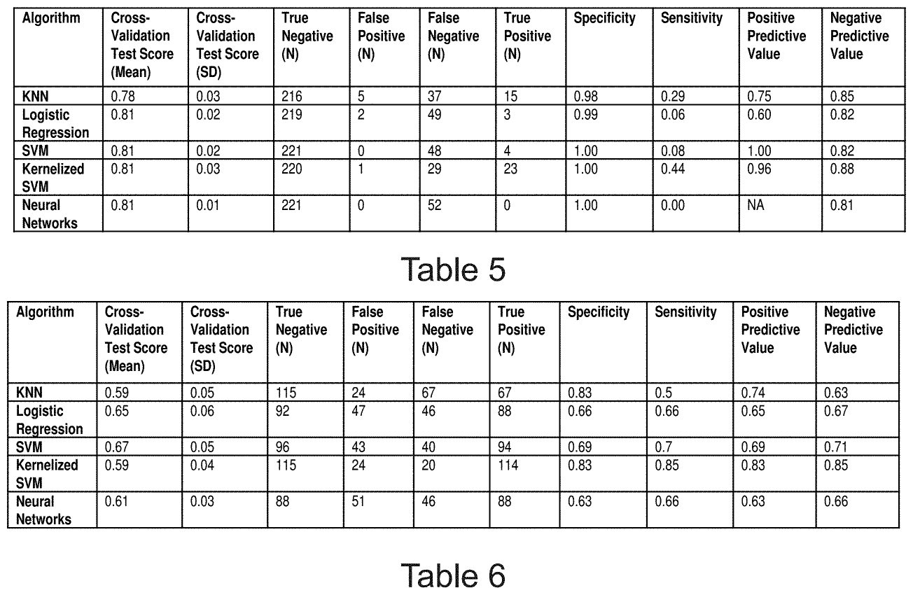

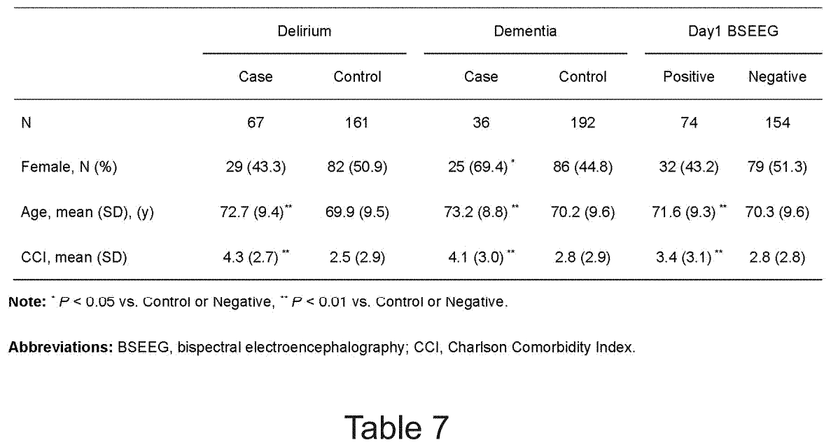

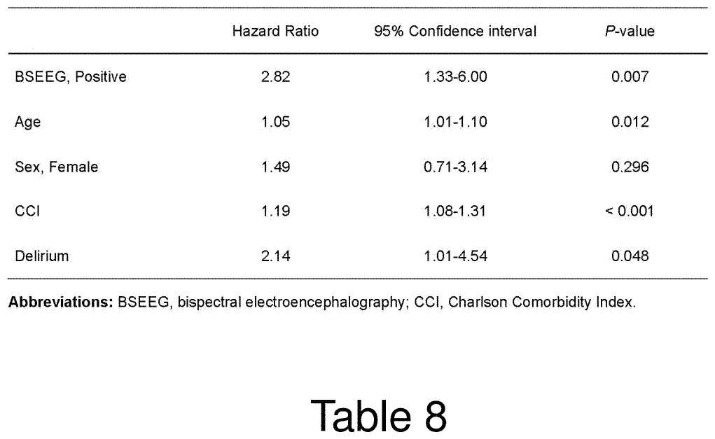

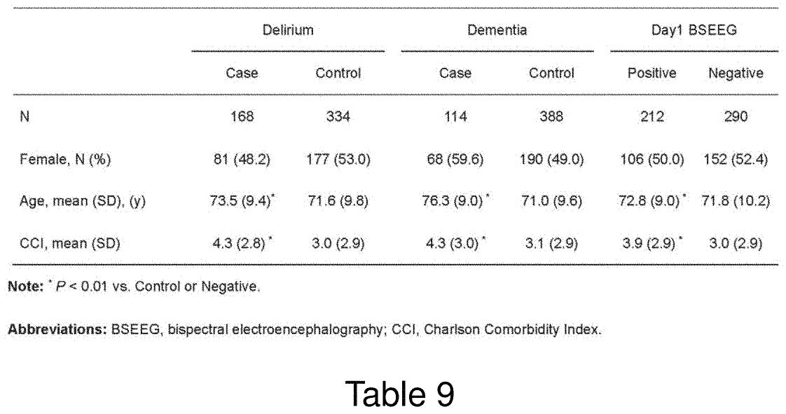

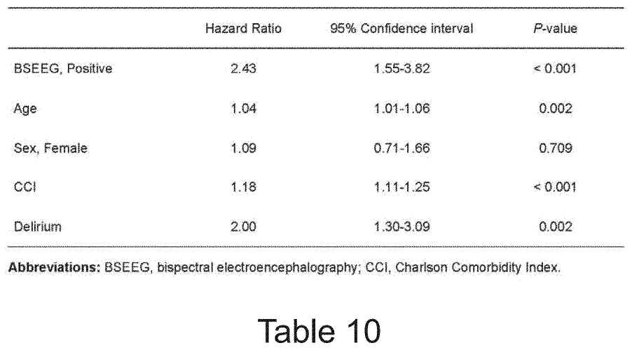

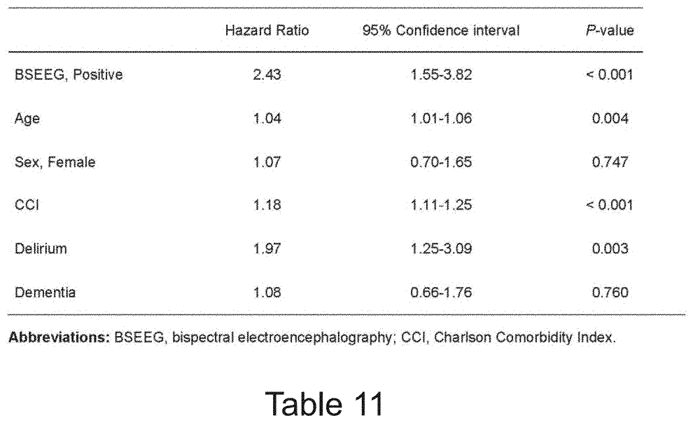

The short-term mortalities were analyzed to show the difference between the patients with and without dementia among total 502 subjects (discovery and replication cohorts). The mortality with dementia in NBSEEG positive group in 60 days was significantly higher than those of NBSEEG negative group (relative risk=3.00; 95% CI, 1.17 to 7.70; p=0.025) as well as those without dementia (relative risk=2.78; 95% CI, 1.46 to 5.28; p=0.001) (FIG. 20). Similarly, the mortality with dementia in NBSEEG positive group in 90 days was significantly higher than those of NBSEEG negative group (relative risk=3.80; 95% CI, 1.52 to 9.48; p=0.002) as well as those without dementia (relative risk=2.39; 95% CI, 1.35 to 4.22; p=0.003) (FIG. 20).

The present study showed the utility of the NBSEEG in predicting mortality with an independent cohort by conducting a replication study. Furthermore, the mortality in patients with dementia who showed high NBSEEG score was higher than those with dementia but with negative NBSEEG score. The result was consistent with our hypothesis that NBSEEG score can predict mortality among dementia patients. To our knowledge, this is the first study that showed the utility of NBSEEG score in predicting mortality with dementia patients.

NBSEEG was shown to be useful in predicting mortality with an independent cohort and a cohort in an increased sample size. Moreover, a score-dependent increase of mortality by the NBSEEG score was replicated as shown in a previous cohort. As it is important to assess a risk of outcome including mortality in elderly inpatients to optimize intervention and care planning, numerous measures to evaluate a risk of mortality have been developed as shown below. For example, the CCI is used for predicting mortality by evaluating comorbidity. Similarly, various measures such as the Multidimensional Prognostic Index (MPI), the Elixhauser comorbidity system, and the single general self-rated health (GSRH) are used for predicting mortality. However, these measures mentioned above have their limitation of lacking biological basis. In addition to the above measures, the NBSEEG score has a potential to be used for predicting mortality as electrophysiological biomarker.

NBSEEG was shown to have utility for predicting mortality was shown for dementia patients as well. This result suggests that we may be able to predict mortality among dementia patients by using NBSEEG score rather than just relying on clinical diagnoses for delirium. Although an appropriate intervention can improve an outcome of patients with delirium, it is well known that detection of delirium in patients with dementia is challenging. Therefore, detection of patients with NBSEEG positive followed by prompt intervention may improve their outcomes whether or not they have dementia.

Importantly, there was a significant difference of mortality even in 30 days between NBSEEG positive and those of negative group. Approximately one in eight with NBSEEG positive died in 30 days, whereas one in thirty-two with NBSEEG negative. It is important to predict a short-term outcome in elderly inpatients because their outcome may be directly related to death. The NBSEEG may be useful for predicting both a short-term and a long-term mortality in elderly inpatients. Furthermore, short-term mortalities and relative risks in NBSEEG positive were higher in patients with dementia compared to those without dementia. Approximately one in three with NBSEEG positive and dementia died in 90 days, whereas one in six with NBSEEG positive but not dementia. These results indicate that the NBSEEG may be useful for patients with dementia to predict a short-term mortality.

The NBSEEG score can predict mortality among elderly patients in general, and even among dementia patients, as soon as 30 days after their hospital admission.

Although the disclosure has been described with reference to preferred embodiments, persons skilled in the art will recognize that changes may be made in form and detail without departing from the spirit and scope of the disclosed apparatus, systems and methods.

What is claimed is:

1. A method for patient screening for outcome risk, comprising:
recording raw BSEEG values via a handheld device;
normalizing the raw BSEEG values to calculate a NBSEEG; and
outputting an outcome NBSEEG score.

2. The method of claim 1, wherein the NBSEEG is calculated by:
comparing the raw BSEEG with a BSEEG population mean; and
dividing the result by the BSEEG population standard deviation.

3. The method of claim 1, wherein the outcome NBSEEG score comprises an NBSEEG positive or NBSEEG negative score.

4. The method of claim 1, wherein the outcome NBSEEG score is continuous.

5. The method of claim 1, wherein the recording is performed at a primary point of care.

6. The method of claim 1, wherein the outcome NBSEEG is correlated with at least one of hospital length of stay (“LOS”), discharge disposition, and/or mortality risk.

7. A handheld system for patient screening for mortality risk, comprising:
a. at least two sensors configured to record one or more brain frequencies;
b. a processor; and
c. at least one module configured to:
i. record raw BSEEG values;
ii. normalize the raw BSEEG values to calculate a NBSEEG; and
iii. output an outcome NBSEEG score.

8. The system of claim 7, wherein the outcome NBSEEG is correlated with at least one of hospital LOS, discharge disposition, and/or mortality risk.

9. The system of claim 7, further comprising outputting threshold data.

10. The system of claim 7, further comprising comparing the outcome NBSEEG score to a threshold.

11. The system of claim 7, further comprising a signal processing device.

12. A method of screening for mortality risk in a subject, comprising:
recording raw BSEEG values from the subject via a handheld device;
normalizing the raw BSEEG values to calculate a NBSEEG; and
outputting an outcome NBSEEG score.

13. The method of claim 12, further comprising comparing the outcome NSBEEG score to a threshold.

14. The method of claim 12, wherein the raw BSEEG values are processed via a signal processing module or feature analysis module in the handheld device.

15. The method of claim 12, wherein the outcome NBSEEG score is categorized as low, medium or high risk by comparison to one or more thresholds.

16. The method of claim 12, further comprising maintaining a BSEEG population norm.

17. The method of claim 16, wherein the NBSEEG is calculated by:
comparing the raw BSEEG with the mean of the BSEEG population norm; and
dividing the result by the BSEEG population standard deviation.

18. The method of claim 17, further comprising recording subject outcome.

19. The method of claim 18, wherein the BSEEG population norm is updated to include the raw BSEEG values and subject outcome.

20. The method of claim 19, wherein the outcome NBSEEG is correlated with at least one of hospital length of stay (“LOS”) and/or discharge disposition.
